(19) United States
(12) Patent Application Publication
(10) Pub. No.: US 2003/0180799 A1
(43) Pub. Date: Sep. 25, 2003

(54) Antibodies against plasma cells

(76) Inventors: Hans Konrad Muller- Hermelink, Wurzburg (DE); Axel Greiner, Triefenstein (DE); Bernd Dorken, Berlin (DE); Ralf Bargou, Berlin (DE); Peter Kufer, Moosburg (DE)

Correspondence Address: Pillsbury Winthrop, Intellectual Property Group, Suite 200, 11682 El Camino Real, San Diego, CA, 92130, US

(21) Appl. No.: 10/168,809

(22) PCT Filed: Dec. 22, 2000
(86) PCT No.: PCT/EP00/13238

(30) Foreign Application Priority Data
Dec. 23, 1999 (DE) 199 62 583.2

(51) Int. Cl. G01N 33/53; C07H 21/04; A61K 48/00; A61K 39/395; C12P 21/02; C12N 5/06; C07K 16/44

(52) U.S. Cl. 435/7.1; 435/69.1; 435/320.1; 435/328; 530/387.3; 536/23.53; 424/132.1; 514/44

(57) ABSTRACT

The present invention relates to a method for producing an antibody, to an antibody specifically reacting with plasma cells, to genes which encode such antibodies, to antigens which are labeled by such an antibody, to additional antibodies directed against said antigens and to methods for producing said antibodies and to uses of such antibodies. In addition, the present invention relates to single-chain multifunctional polypeptides comprising (a) a first domain comprising a binding site of the antibodies defined herein and (b) a second domain comprising a binding site of an immunoglobulin chain or an antibody specifically recognizing the CD3 antigen. Furthermore, compositions and kits comprising the compounds of the invention are disclosed. Preferably said compositions are pharmaceutical or diagnostic compositions.

The present invention relates to a method for producing an antibody against plasma cells, to an antibody, to genes which encode such antibodies, to antigens which are labeled by such an antibody, to additional antibodies directed against said antigens and to methods for producing said antibodies and to uses of such antibodies. In addition, the present invention relates to single-chain multifunctional polypeptides comprising (a) a first domain comprising a binding site of the antibodies defined herein and (b) a second domain comprising a binding site of an immunoglobulin chain or an antibody specifically recognizing the CD3 antigen. Such antibodies and/or single chain multifunctional polypeptides are required, in particular, in the field of biological and medical diagnostics and in the therapy of autoimmune diseases or tumors such as plasmocytomas or lymphomas. The invention also provides for nucleic acid molecules/polynucleotides encoding for said antibodies and/or single chain multifunctional polypeptides, as well as for vectors and host cells comprising said nucleic acid molecules/polynucleotides. Finally, the present invention provides for compositions comprising the compounds of the present invention. Preferably said compositions are pharmaceutical and/or diagnostic compositions.

In animal organisms and in humans, too, the immune system responds to stimuli (antigens) in two different ways. On the one hand, it produces a humoral immune response, wherein antibodies are produced by plasma cells (differentiated B-lymphocytes) which are directed against the antigen recognized as a foreign substance. On the other hand, it may produce a cellular immune response via T-lymphocytes, which in turn stimulate B-cells.

The antibodies which are produced by the humoral immune response are directed against an antigenic determinant, that is to say a specific region (epitope) on the antigen and bind thereto. These antibodies are globular proteins (immunoglobulins Ig, for instance IgM or IgG) consisting of two identical chains each, a heavy chain (H-chain) and a light chain (L-chain), with each one of the L-chain being connected to one H-chain via disulfide bridges and each such pair forming a specific binding site for the antigenic determinant. Antibodies against any antigens are produced in a conventional manner such that an animal organism is inoculated with the antigen and its humoral immune response subsequently forms antibodies against the inoculated antigen. These antibodies can subsequently be isolated. Moreover, it is nowadays possible to produce so-called monoclonal antibodies. For this purpose, a proliferating myeloma cell is fused with a B-cell from an immunized animal organism, which produces the corresponding antibody, as the B-cell. As a result, hybridoma cells are formed, which proliferate just as the myeloma cells, on the one hand, and form the antibody just as the plasma cell, on the other hand. These hybridoma cells are cultured and obtained from the culture of the monoclonal antibodies produced. Regarding these generally known and frequently used methods, attention is drawn, for instance, to Greiner A. et al. Laboratory Investigation (1994), vol. 70, pages 572-578, and the other source indications contained therein.

Thus, the monoclonal antibody technique allows identical antibodies to be produced in great numbers.

During the differentiation of B-cells into antibody-producing plasma cells various disorders may occur resulting in serious diseases in humans and animals. On the B-cell level, endogenic cells may be wrongly detected by antibodies, thereby producing, for instance, autoimmune diseases, wherein large numbers of antibodies against endogenic cells are produced and secreted by corresponding plasma cells. On the other hand, it is possible that B-cells start to divide in an uncontrolled manner, thereby forming tumors (lymphomas). Moreover, such an unlimited proliferation is also known on the plasma cell level (syndromes: plasmocytoma, multiple myeloma).

Multiple myeloma is a plasma cell disorder characterized by the accumulation of malignant plasma cells in the bone marrow and the increased production of a specific immunoglobulin, usually monoclonal IgG or IgA. Common complications of overt multiple myeloma include recurrent bacterial infections, anemia, osteoloytic lesions, and renal insufficiency. Multiple myeloma is responsible for about 1 percent of all cancer-related deaths in Western countries. Its epidemiologic pattern remains obscure, and ist cause is unknown (Bataille and Harousseau, 1997, N. Engl. J. Med. 336).

In order to treat the disease, it is necessary to immunologically detect the participating degenerated cells.

It is true that the prior art describes antibodies against plasma cells which recognize all plasma cells. However, the antigen corresponding to these antibodies is only present intracellularly, and therefore it is necessary to destroy the cells prior to detection (see Anderson et al. (1984) J. Immunol., vol. 132, 3271-3179). Hence, it is not possible to detect plasma cells without destroying them.

Moreover, antibodies against surface antigens are known (see Anderson et al. (1984) J. Immunol., vol. 132, 3172-3179, Anderson et al. (1983) J. Immunol., vol. 130, 1132-1138, Tong et al. (1987) Blood, vol. 69, 238-148 or Turley et al. (1994), J. Clin. Pathol., vol. 47, 418-422). However, these antibodies have no specificity and detect both immature precursor cells and mature plasma cells. According to these investigations, plasma cells in the differentiated, antibody-secreting stage lose the typical surface antigens of the B-cell stage. Therefore, it is not possible to sort out immature precursor cells (bone marrow cells or B-cells) from mature cells (differentiated plasma cells which form antibodies themselves).

Consequently, it has so far not been possible to detect specific surface molecules (cluster of differentiation, CD) which could serve as antibody receptors (antigenic determinants). Due to their completely differentiated condition, intact plasma cells are immunologically hardly recognizable or distinguishable.

Hence, a therapy of the afore-mentioned syndromes which would specifically attack the mature plasma cells is not known.

Thus, the only therapy so far available for the above-mentioned syndromes is chemotherapy, in particular in the form of the multimode chemotherapy. However, it suffers from the generally known drawbacks of a chemotherapy. An overview of the therapy concepts so far known is for instance contained in Lokhorst H. M. et al., Br. J. Haematol. (1999), vol. 106, pages 18-27.

Despite some progress with the above mentioned high dose chemotherapy and autologous stem cell/bone marrow transplantation multiple myeloma is still an incurable disease with a median survival of about 3 to 5 years (Peest (1995) Eur J Cancer 31 a:146-151; Attal (1996) N Engl J Med 335:91-97). This bleak situation has stimulated the search for alternative therapeutic strategies, among which immunotherapeutic strategies are playing an increasing role. The development of antibody-based strategies for the removal of plasma cells in general and the treatment of multiple myeloma in particular have been hampered by the fact that suitable plasma cell-specific surface antigens are missing so far (Bataille and Harousseau, 1997, loc. cit.; Hallek, 1998 Blood 91, 3-21; see also Greiner, 2000, Virchows Arch 437, 372-379). Even if much research work regarding B-cell differentiation in the initial stages of B-cell development has been done so far, little is known about the terminal differentiation on the way to the plasma cell. The reason therefor is essentially that antibodies specific to plasma cells are yet not available for their identification. Antibodies, such as CD44, CD38, PC-1, PCA-1, MMA, BB-1 or VS38, so far published for plasma cells, have the disadvantage of a wide reaction range and of also reacting with various other tissues and with cytoplasmic antigens. Although normal and malignant plasma cells express a number of well characterized surface markers they all have turned out to be non plasma cell-specific. CD38 is an activation rather than a differentiation-associated antigen and lacks lineage restriction (Funaro, 1990; Alessio, 1990, J. Immunol. 145, 878-884). CD56 is a N-CAM splice variant also expressed on NK-cells (Pellat-Deceunynck et al., 1998, Leukemia 12, 1977-1982; Robillard, 1998, Clin. Cancer Res. 4, 1521-1526), 3). CD138 (syndecan-1) is also expressed on epithelia (Sebestyen, 1999, Br. J. Haematol. 104, 412-419; Maatta, 1999, J. Biol. Chem. 274, 9891-9898; Anttonen, 1999, Br. J. Cancer 79, 558-564; Kato, 1995, Mol. Biol. Cell 6, 559-576). A recently described plasma cell associated antigen HM1.24 has turned out to be also expressed by other cell types such as bone marrow stromal cells (Bataille and Harousseau, 1997, loc. cit.; Hallek, 1998, loc. cit.; Greiner, 2000, loc. cit.; Ohtomo, 1999, Biochem. Biophys. Res. Commun. 258, 583-591). Thus all of the above described antibodies do not specifically identify plasma cells and multiple myeloma cells.

Antibody-based approaches to eliminate lymphoma cells have proven to be effective in man either in the form of standard IgG antibodies as well as radio-immunoconjugates. Those molecules are based on antibodies recognizing CD20 and CD52 (rituximab, ibritumomab, tositumomab, Campath-1H). Although these antibody-based approaches show very promising results in lymphoma and leukemia therapies, the targets are not suitable for plasma cell malignancies (plasmacytoma/multiple myeloma).

Plasma cells constitute only one percent of all B-cells and only a small proportion express the pan-B cell markers CD19 (usually CD19 negaitve) and CD20 (less than 20%). Thus therapeutic approaches based on antibodies directed against CD19 or CD20 will only target a small subset of plasma cells, while inducing a major cytotoxic response due to the undesired elimination of non-plasma B cells.

Consequently, the present invention addresses the problem of proposing a method for preparing antibodies and of proposing antibodies which specifically label the plasma cells. Moreover, the present invention addresses the problem of proposing genes encoding corresponding antibodies and uses of such antibodies. Furthermore, it addresses the problem of proposing an antigen labeled with such antibodies. In addition, the technical problem of the present invention was to provide for means and methods to alleviate, prevent or cure plasma cells malignancies.

The problem is solved by the antibody, the single-chain polypeptide, the antigen nucleotide sequences encoding said antibody, single-chain polypeptides or antigen, the uses and the methods for preparing additional antibodies and/or single-chain polypeptides of the present invention. Advantageous embodiments are specified in the dependent claims.

In order to obtain antibodies against a specific antigen, the state of the art proposes to inoculate animal organisms with the corresponding antigen (for instance by injection), with the result that they form antibodies against this antigen on account of their humoral immune response. The antibodies produced can subsequently be separated and purified. So far, this method has not been successful with intact mature plasma cells, as they have few antigenic determinants on their surface, with which it would have been possible to prepare specific antibodies specifically for plasma cells. Attempts of carrying out inoculations with plasma cells were not successful therefor.

This is the starting point of the present invention, wherein in contrast to the prevailing teaching, an animal organism is immunized with B-cells of the plasma differentiation line up to and including the lymphoplasmacytoid cell stage. Hence, in contrast to the prior art, it is not the desired antigen, i.e. the plasma cells, but their precursors that are used for inoculation. However, surprisingly, this very method allows antibodies to be obtained which specifically label plasma cells without labeling the corresponding precursor stages. The reason therefor may be that the corresponding precursor cells after inoculation develop further into plasma cells in the animal organism where they trigger a specific antibody reaction, which cannot be produced by direct immunization with plasma cells. From such an immunized animal organism it is now readily possible to obtain the produced antibodies as monoclonal antibodies by fusing corresponding spleen cells from said animal organism with myeloma cells into hybridoma cells. In this respect, reference is made to the generally known state of the art for producing monoclonal antibodies (see also herein below).

Antibodies are essentially defined by the variable regions (Fw) of their L- and H-chains. The other regions (F_{C}) of the L-chain and H-chain, respectively, make no contribution to the antigen specificity of the antibody and are in each case largely invariable in different antibody classes.

The antibodies of the invention are characterized in that the variable region (Fw(L)) of at least one of its light chains (L-chain) possesses at least one of the following amino acid sequences or a part of it.

| DIVMTQTPLTLSVTIGQPASLSC, | (variable region FW-1; SEQ ID NO: 1) |
|---|---|
| KSSQSLLDSDGKTYLN, | (complementarity-determining region CDR-1; SEQ ID NO: 2) |
| WLLQRPGQSPKRLIS, | (variable region FW-2; SEQ ID NO: 3) |
| LVSKLDS, | (complementarity-determining region CDR-2; SEQ ID NO: 4) |
| GVPDRFTGSGSGTDFTLKISRVEAEDLGVYYC, | (variable region FW-3; SEQ ID NO: 5) |
| WQGTHLPWT | (complementarity-determining region CDR-3; SEQ ID NO: 6) |
| and/or | |
| FGGGTKLEIKR | (variable region FW-4; SEQ ID NO: 7) |
| and/or the variable region (Fw(H)) of at least one of its heavy chains (H-chain) | |
| possesses at least one of the following amino acid sequences or a part thereof: | |
| QVQLQQSGPELVKTGASVKISCKGSGYSFS, | (variable region FW-1; SEQ ID NO: 8) |
| GYYMH | (complementarity-determining region CDR-1; SEQ ID NO: 9) |
| WVKQSHGKRLEWIG, | (variable region FW-2; SEQ ID NO: 10) |
| YISGYNGDTRYNQKFRG, | (complementarity-determining region CDR-2; SEQ ID NO: 11) |
| KATFIVDISSRTAYMQFNSLTSEDSAVYYCAR | (variable region FW-3; SEQ ID NO: 12) |
| GGYYGYVDY, | (complementarity-determining region CDR-3; SEQ ID NO: 13) |
| and/or | |
| WGQGTTLTVSS. | (variable region FW-4; SEQ ID NO: 14) |

These antibodies can also be characterized in that the gene encoding the individual chains (L-and H-chain) of the antibody contains the following nucleotide sequences or corresponding fragments thereof:

for the L-chain
(SEQ ID NO: 15)
gaagcacgcg tagatatcgt gatgacccaa actccactca ctttgtcggt taccattgga caaccagcct
ccctctcttg caagtcaagt cagagcctct tagatahtga tggaaagaca tatttgaatt ggttgttaca
gaggccaggc cagtctccaa agcgcctaat ctctctggtg tctaaattgg actctggagt ccctgacaga
ttcactggca gtggatcagg gacagatttc acactgaaaa tcagcagagt ggaggctgag gatttgggag
tctattattg ctggcaaggt acacatcttc cgtggacatt cggtggaggc accaagctgg aaatcaaacg
ggctgatgct gcggccgctg gatccatctt c
and/or for the H-chain
(SEQ ID NO: 16)
cgccatggcc gcgggattcc ggccatggcg caggtgcagc tgcagcagtc tggacctgag ctagtgaaga
ctggggcttc agtgaagata tcttgtaagg gttctggtta ctcattcagt ggttactaca tgcactgggt
caagcagagc catggaaaga ggcttgagtg gattggatat attagtggtt ataatggtga tactaggtat
aatcagaagt tcaggggcaa ggccacattt attgtagaca tatcctccag gacagcctac atgcagttca
acagcctgac atctgaagac tctgcggtct attactgtgc aagagggggt tactacggct acgtggacta
ctggggccaa ggcaccaccc tcacagtctc ctcagccaaa acgacaccca agcttgtcta tccactggcc
cctggtaatc actgtgcggc cgccg

The nucleotide sequence according to the invention in each case contains the above-mentioned nucleotide sequences or a part thereof and in each case represents one of the genes necessary for the above-mentioned antibody, functional antibody, fragment and/or functional derivative thereof.

The antibody may be a conventional immunoglobulin, for instance an immunoglobulin G (IgG), in which both light chains and both heavy chain each have the same amino acid sequence. Preferably said antibody is a mouse IgG2 or a human IgG1, yet other IgGs are also envisaged. However, the invention also covers antibodies which are multifunctional polypeptides and/or bispecific, and for instance contain only one heavy and one light chain as described above, with the result that only one binding site for the plasma cells is formed. Particularly preferred bispecific antibodies are described herein below. It is particularly preferred that said bispecific antibodies are single-chain constructs.

The antibody of the invention can therefore be, e.g., a monoclonal antibody, polyclonal antibody, chimeric antibody, humanized antibody, bispecific antibody, synthetic antibody, antibody fragment such as Fab, Fv or scFv fragments etc., or a chemically modified derivative of any of these. Monoclonal antibodies can be prepared, for example, by the techniques as originally described in Kohler and Milstein, Nature 256 (1975), 495, and Galfre, Meth. Enzymol. 73 (1981), 3, which comprise the fusion of mouse myeloma cells to spleen cells derived from immunized mammals with modifications developed in the art. Monoclonal antibodies can, inter alia, be obtained by immunizing mice, for example BALB/c mice with human mononuclear blood cells obtainable as described in appended example 1. Furthermore, additional antibodies or fragments thereof to the aforementioned plasma cells can be obtained by using methods which are described, e.g., in Harlow and Lane “Antibodies, A Laboratory Manual”, CSH Press, Cold Spring Harbor, 1988. The antibodies of the invention can be used, for example, for the immunoprecipitation, immunolocalization or purification of plasma cells of the invention as well as for the monitoring of the presence of such plasma cells and for the identification of compounds interacting with the plasma cells according to the invention. For example, surface plasmon resonance as employed in the BIAcore system can be used to increase the efficiency of phage display of antibodies which bind to an epitope recognized by an antibody of the invention (Schier, Human Antibodies Hybridomas 7 (1996), 97-105; Malmborg, J. Immunol. Methods 183 (1995), 7-13). The production of chimeric antibodies is described, for example, in WO89/09622. Methods for the production of humanized antibodies are described in, e.g., EP-Al 0 239 400 and WO90/07861. Furthermore, human antibodies in general have become accessible since the availability of transgenic mice expressing human antibodies also called xenogenic antibodies (Brüggemann, Immunol. Today 17 (1996), 391-397; the general principle for the production of xenogenic antibodies such as human antibodies in mice is described in, e.g., WO 91/10741, WO 94/02602, WO 96/34096 and WO 96/33735) and of the combinatorial antibody library and phage display technology allowing the in vitro combination of variable regions of immunoglobulin heavy and light chains (V_{H} and V_{L}) and the in vitro selection of their antigen binding specificity (Winter, Annu. Rev. Immunol. 12 (1994), 433-455). By using the phage display method, rare events like one specific binding entity out of 10⁷ to 10⁹ different V_{L}/V_{H}- or V_{H}/V_{L}-pairs can easily be isolated; this is especially true when the repertoire of variable regions has been enriched for specific binding entities by using B-lymphocytes from immunized hosts as a source for repertoire cloning. In addition, approaches using semisynthetic or fully synthetic V_{H}- and/or V_{L}-immunoglobulin chain repertoires have been developed. For example, almost the complete repertoire of unrearranged human V-gene-segments has been cloned from genomic DNA and used for in vitro recombination of functional variable region genes, resembling V-J- or V-D-J-recombination in vivo (Hoogenboom, J. Mol. Biol. 227 (1992), 381-388; Nissim, EMBO J. 13 (1994) 692-698; Griffiths, EMBO J. 13 (1994), 3245-3260). Hence, all these derivatives of the antibody described herein below and in the appended examples are within the scope of the present invention as long the antibody recognizes specifically at least one epitope of an antigen specific for plasma cells, preferably human plasma cells. As discussed herein, the antibody of the invention may exist in a variety of forms besides complete antibodies; including, for example, Fv, Fab and F(ab)₂, as well as in single chains; see e.g. WO88/09344 and herein below.

The antibodies of the invention, their corresponding immunoglobulin chain(s) and/or functional fragments and derivatives thereof can be further modified using conventional techniques known in the art, for example, by using amino acid deletion(s), insertion(s), substitution(s), addition(s), and/or recombination(s) and/or any other modification(s) known in the art either alone or in combination. Methods for introducing such modifications in the DNA sequence underlying the amino acid sequence of an immunoglobulin chain are well known to the person skilled in the art; see, e.g., Sambrook, Molecular Cloning A Laboratory Manual, Cold Spring Harbor Laboratory (1989) N.Y.

In a preferred embodiment of the present invention, the antibody of the invention is the antibody which is produced by the hybridoma cell line DSM ACC 2441, preferably said antibody is the antibody WUE-1 that is produced by said hybridoma cell line.

Said hybridoma cell has been deposited in the culture collection Deutsche Sammlung von Mikroorganismen und Zellkulturen (DSMZ) GmbH in Braunschweig, Germany on Jan. 1, 2000, in accordance with the Budapest Treaty. The antibodies as described herein are particularly useful, inter alia, for the immuno-isolation, immuno-localization and/or purification of plasma cells. Furthermore, they might be employed in assays for the detection and/or identification of compounds which are capable of interacting with or which are interacting with plasma cells.

The present invention also relates to a (human) B cell line which is capable of producing the antibody of the invention in a humanized form, e.g., by transduction of the cDNA coding for the variable heavy and light chain domains of WUE-1 antibody linked to the constant domains of an immunoglobulin into a human B cell line. Said cDNA is obtainable by methods known to the person skilled in the art and are described, inter alia, in Sambrook, loc. cit. and Ausubel “Current Protocols in Molecular Biology”, Green Publishing Associates and Wiley Interscience, N.Y. (1989). The cloning of said cDNA for expression (or sequencing) may follow standard protocols as described, e.g., in Orlandi, PNAS 86 (1989), 3833-3837 or as illustrated in appended examples, documenting the cloning of the variable regions of mAb WUE-1.

Furthermore, the present invention provides for a nucleotide sequence/polynucleotide encoding at least a variable region of an antibody of the invention and/or of an immunoglobulin chain of any of the before described antibodies of the invention. Polynucleotides encoding said regions are obtainable by methods which are well known in the art and comprise, inter alia, cloning techniques as described in Orlandi, PNAS 86 (1989), 3833-3837 or Sambrook, loc. cit. One form of immunoglobulin constitutes the basic structural unit of an antibody. This form is a tetramer and consists of two identical pairs of immunoglobulin chains, each pair having one light and one heavy chain. In each pair, the light and heavy chain variable regions or domains are together responsible for binding to an antigen, and the constant regions are responsible for the antibody effector functions. In addition to antibodies, immunoglobulins may exist in a variety of other forms (including less than full-length that retain the desired activities), including, the above mentioned Fv, Fab, and F(ab′)2, as well as single chain antibodies (e.g., Huston, Proc. Nat. Acad. Sci. USA 85(1988, 5879-5883 and Bird, Science 242(1988), 423-426); see also herein below. An immunoglobulin light or heavy chain variable domain consists of a “framework” region interrupted by three hypervariable regions, also called CDRs. The extent of the framework region and CDRs have been precisely defined; see, e.g., “Sequences of Proteins of Immunological Interest,” Kabat, U.S. Department of Health and Human Services (1990). The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen. Chimeric antibodies are antibodies whose light and heavy chain genes have been constructed, typically by genetic engineering, from immunoglobulin variable and constant region genes belonging to different species. For example, the variable segments of the genes from a mouse monoclonal antibody may be joined to human constant segments.

Therefore, the teachings of the present invention provide for variable regions, in particular for CDRs of antibodies specifically detecting/interacting with plasma cells. Said variable regions may, inter alia, be employed to generate, via known recombinant techniques, chimeric, synthetic, etc. antibodies and/or immunoglobulin molecules or derivatives thereof. It is, for example, envisaged that the variable regions of the antibodies of the present invention be combined with constant regions of antibodies of a different Ig-subtype or of an Ig/Ig-subtype of a different species. This may, inter alia, particularly be useful in enhancing the effector function of said antibody, e.g. the effector function of induction of antibody-depedent cell-mediated cytotoxicity (ADCC) and/or complement-dependent cytotoxicity. The skilled artisan is aware that such effector functions largely depend on the constant part of the antibody/immunoglobulin molecule and would chose such a constant part accordingly.

Thus, the antibodies of the present invention can be produced by expressing recombinant DNA segments encoding the heavy and light immunoglobulin chain(s) of the antibody invention either alone or in combination. Said polynucleotide may be, e.g., DNA, cDNA, RNA or synthetically produced DNA or RNA or a recombinantly produced chimeric nucleic acid molecule comprising any of those polynucleotides either alone or in combination. Preferably said polynucleotide is part of a vector. Such vectors may comprise further genes such as marker genes which allow for the selection of said vector in a suitable host cell and under suitable conditions. Preferably, the polynucleotide of the invention is operatively linked to expression control sequences allowing expression in prokaryotic or eukaryotic cells. Expression of said polynucleotide comprises transcription of the polynucleotide into a translatable mRNA. Regulatory elements ensuring expression in eukaryotic cells, preferably mammalian cells, are well known to those skilled in the art. They usually comprise regulatory sequences ensuring initiation of transcription and optionally poly-A signals ensuring termination of transcription and stabilization of the transcript. Additional regulatory elements may include transcriptional as well as translational enhancers, and/or naturally-associated or heterologous promoter regions. In this respect, the person skilled in the art will readily appreciate that the polynucleotides encoding at least the variable domain of the light and/or heavy chain may encode the variable domains of both immunoglobulin chains or only one. Likewise, said polynucleotides may be under the control of the same promoter or may be separately controlled for expression. Possible regulatory elements permitting expression in prokaryotic host cells comprise, e.g., the P_{L}, lac, trp or tac promoter in E. coli, and examples for regulatory elements permitting expression in eukaryotic host cells are the AOX1 or GALl promoter in yeast or the CMV-, SV40-, RSV-promoter (Rous sarcoma virus), CMV-enhancer, SV40-enhancer or a globin intron in mammalian and other animal cells. As documented in the appended examples for the antibody derivative, e.g. a single-chain construct, the EF-2 promoter may be employed. Beside elements which are responsible for the initiation of transcription such regulatory elements may also comprise transcription termination signals, such as the SV40-poly-A site or the tk-poly-A site, downstream of the polynucleotide. In this context, suitable expression vectors are known in the art such as Okayama-Berg cDNA expression vector pcDV1 (Pharmacia), pCDM8, pRc/CMV, pcDNA1, pcDNA3 (In-vitrogene), pSPORT1 (GIBCO BRL). Preferably, the expression control sequences will be eukaryotic promoter systems in vectors capable of transforming or transfecting eukaryotic host cells, but control sequences for prokaryotic hosts may also be used. Once the vector has been incorporated into the appropriate host, the host is maintained under conditions suitable for high level expression of the nucleotide sequences, and, as desired, the collection and purification of the immunoglobulin light chains, heavy chains, light/heavy chain dimers or intact antibodies, binding fragments or other immunoglobulin forms may follow; see, Beychok, Cells of Immunoglobulin Synthesis, Academic Press, N.Y., (1979).

As described above the nucleotide sequence polynucleotide of the invention can be used alone or as part of a vector to express the antibody of the invention in cells, for, e.g., gene therapy or diagnostics of diseases related to malignancies of plasma cells. The polynucleotides or vectors containing the DNA sequence(s) encoding any one of the above described antibodies is introduced into the cells which in turn produce the antibody of interest. Gene therapy, which is based on introducing therapeutic genes into cells by ex-vivo or in-vivo techniques is one of the most important applications of gene transfer. Suitable vectors, methods or gene-delivery systems for in-vitro or in-vivo gene therapy are described in the literature and are known to the person skilled in the art; see, e.g., Giordano, Nature Medicine 2 (1996), 534-539; Schaper, Circ. Res. 79 (1996), 911-919; Anderson, Science 256 (1992), 808-813; Isner, Lancet 348 (1996), 370-374; Muhlhauser, Circ. Res. 77 (1995), 1077-1086; Wang, Nature Medicine 2 (1996), 714-716; WO94/29469; WO 97/00957, Onodua, Blood 91 (1998), 30-36; Verzeletti, Hum. Gene Ther. 9 (1998), 2244-2251; Verma, Nature 389 (1997), 239-242; U.S. Pat. No. 5,580,859; U.S. Pat. No. 5,589,466; U.S. Pat. No. 4,394,448 or Schaper, Current Opinion in Biotechnology 7 (1996), 635-640, and references cited therein. The polynucleotides and vectors of the invention may be designed for direct introduction or for introduction via liposomes, or viral vectors (e.g. adenoviral, retroviral) into the cell. Preferably, said cell is a germ line cell, embryonic cell, or egg cell or derived therefrom, most preferably said cell is a stem cell. This embodiment is particularly suited for bispecific antibodies of the invention, e.g., with one specificity against a plasma cell antigen which would facilitate the treatment of plasma cell related diseases described herein (see also appended examples).

Furthermore, the present invention relates to vectors, particularly plasmids, cosmids, viruses and bacteriophages used conventionally in genetic engineering that comprise a polynucleotide encoding a variable domain of a chain of an antibody of the invention; optionally in combination with a polynucleotide of the invention that encodes the variable domain of the other chain of the antibody of the invention. Preferably, said vector is an expression vector and/or a gene transfer or targeting vector. Expression vectors derived from viruses such as retroviruses, vaccinia virus, adeno-associated virus, herpes viruses, or bovine papilloma virus, may be used for delivery of the polynucleotides or vector of the invention into targeted cell population. Methods which are well known to those skilled in the art can be used to construct recombinant viral vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1989). Alternatively, the polynucleotides and vectors of the invention can be reconstituted into liposomes for delivery to target cells. The vectors containing the polynucleotides of the invention (e.g., the heavy and/or light variable domain(s) of the immunoglobulin chains encoding sequences and expression control sequences) can be transferred into the host cell by well-known methods, which vary depending on the type of cellular host. For example, calcium chloride transfection is commonly utilized for prokaryotic cells, whereas calcium phosphate treatment or electroporation may be used for other cellular hosts; see Sambrook, supra. Once expressed, the whole antibodies, their dimers, individual light and heavy chains, or other immunoglobulin forms of the present invention, can be purified according to standard procedures of the art, including ammonium sulfate precipitation, affinity columns, column chromatography, gel electrophoresis and the like; see, Scopes, “Protein Purification”, Springer-Verlag, N.Y. (1982). Substantially pure immunoglobulins of at least about 90 to 95% homogeneity are preferred, and 98 to 99% or more homogeneity most preferred, for pharmaceutical uses. Once purified, partially or to homogeneity as desired, the polypeptides may then be used therapeutically (including extracorporeally) or in developing and performing assay procedures.

The present invention furthermore relates to host cells transformed with a nucleotide sequence/polynucleotide or vector of the invention. Said host cell may be a prokaryotic or eukaryotic cell. The polynucleotide or vector of the invention which is present in the host cell may either be integrated into the genome of the host cell or it may be maintained extrachromosomaly.

The host cell can be any prokaryotic or eukaryotic cell. The term “prokaryotic” is meant to include all bacteria which can be transformed or transfected with a DNA or RNA molecules for the expression of an antibody of the invention or the corresponding immunoglobulin chains. Prokaryotic hosts may include gram negative as well as gram positive bacteria such as, for example, E. coli, S. typhimurium, Serratia marcescens and Bacillus subtilis. The term “eukaryotic” is meant to include but not being limited to insect, fungal, plant, animal or human cells. Preferred fungal cells are, for example, those of the genus Saccharomyces, in particular those of the species S. cerevisiae. A polynucleotide coding for an antibody of the invention can be used to transform or transfect the host using any of the techniques commonly known to those of ordinary skill in the art. Especially preferred is the use of a plasmid or a virus containing the coding sequence of the antibody recognizing plasma cells, e.g. and epitope of the antigen of the invention for purposes of eukaryotic or prokaryotic transformation or transfection, respectively. Methods for preparing fused, operably linked genes and expressing them in, e.g., mammalian cells and bacteria are well-known in the art (Sambrook, et al., Molecular Cloning: A Laboratory Manual, Cold Spring Harbor Laboratory, Cold Spring Harbor, N.Y., 1989). The genetic constructs and methods described therein can be utilized for expression of the antibody of the invention in eukaryotic or prokaryotic hosts. In general, expression vectors containing promoter sequences which facilitate the efficient transcription of the inserted polynucleotide are used in connection with the host. The expression vector typically contains an origin of replication, a promoter, and a terminator, as well as specific genes which are capable of providing phenotypic selection of the transformed cells. The transformed hosts can be grown in fermentors and cultured according to techniques known in the art to achieve optimal cell growth. The antibody or its corresponding immunoglobulin chain(s) of the invention can then be isolated from the growth medium, cellular lysates, or cellular membrane fractions. The isolation and purification of the, e.g., microbially expressed antibodies or immunoglobulin chains of the invention may be by any conventional means such as, for example, preparative chromatographic separations and immunological separations such as those involving the use of monoclonal or polyclonal antibodies directed, e.g., against the constant region of the antibody of the invention.

In a further embodiment the present invention relates to a continuous, stable antibody-producing cell line which is capable of producing an antibody or a functional fragment or derivative thereof of the invention. Said cell line may be, as discussed herein above, a hybridoma cell line, preferably the hybridoma cell line having the deposit number DSM ACC 2441.

In yet another embodiment, the present invention provides for a method for preparing an antibody against plasma cells, characterized in that an animal is immunized with B-cells of the plasma cell differentiation line up and including the lymphoplasmacytoid cell stage and the prepared antibody is isolated in a conventional manner from the blood of the animal. As, inter alia, illustrated in the appended examples, mice, preferably BALB/c mice may be immunized according to standard protocols with, e.g. a lymphoma cell line, inter alia a lymphoma of the MALT-type (H3302), capable of differentiating into a plasma cell in vitro.

Furthermore, the invention provides for preparing an antibody or a functional fragment or derivative thereof capable of recognizing plasma cells comprising

(a) culturing the (host) cell or hybridoma cell of the present invention, capable of expressing an antibody, functional fragment, derivative thereof or an immunoglobulin chain and

(b) isolating said antibody, functional fragment, derivative or immunoglobulin chain thereof from the cells or the culture medium.

The present invention also involves a method for producing cells capable of expressing an antibody of the invention or its corresponding immunoglobulin chain(s) comprising genetically engineering cells with the polynucleotide or with the vector of the invention. Preferably, the immunoglobulin chain(s) thus expressed are displayed on the cell surface of the transfected cell. This embodiment as well as some others mentioned herein may be adapted for phage display techniques such as described above. The cells obtainable by the method of the invention can be used, for example, to test the interaction of the antibody of the invention with its antigen. The cells obtainable by the above-described method may also be used for the screening methods referred to herein below. Furthermore, transgenic animals, preferably mammals, comprising a polynucleotide, vector or cells of the invention may be used for the large scale production of the antibody of the invention.

Furthermore, the invention relates to an antibody of the invention or a fragment or a derivative thereof or immunoglobulin chain encoded by a polynucleotide according to the invention or obtainable by the above-described method or from cells produced by the method described above.

In this context it is also understood that the antibodies, fragments and/or derivatives thereof according to the invention may be further modified by conventional methods known in the art. By providing the antibodies according to the present invention it is also possible to determine the portions relevant for their binding activity. This may allow the construction of chimeric proteins comprising an amino acid sequence derived from an antibody of the invention which is crucial for binding activity and other functional amino acid sequences e.g. nuclear localization signals, transactivating domains, DNA-binding domains, hormone-binding domains, protein tags (GST, GFP, h-myc peptide, Flag, HA peptide) which may be derived from heterologous proteins.

The antibodies of the invention can also be present as antibody conjugates, for instance with proteins, such as toxins, cytokines, antibody fragments or enzymes, and/or with fluorophores, biotin and/or radioisotopes. Such antibody conjugates and their preparation are described for instance in “G. T. Hermanson “Bioconjugate Techniques” Academic Press ISBN 0-12-342336-8, 1995”, in particular in chapters 8, 10, 11 and 13, or in “J. E. Coligan et al. “Current Protocols in Immunology” J. Wiley and Sons, Inc. ISBN 0-471-52276-7, 1991”, all of which are incorporated by reference into the disclosure content of this application.

Conjugation of radioisotopes is mediated by hetero-bifunctional chelate-forming active ingredients (see above, under G. T. Hermanson, Chapter 8).

Suitable radioisotopes include yttrium-88, yttrium-90, indium-111, iodine-125, iodine-131, samarium-153, lutetium-177, rhenium-186, bismuth-212 or bismuth-213.

Suitable toxins include ricin, recombinant ricin, bacterial toxins PE (Pseudomonas Exototoxin) or DT (diphtheria toxin) which are single-chain toxins lending themselves to the preparation of recombinant fusion toxins.

Antibodies which are conjugated with toxic antibiotics are disclosed, for instance, in U.S. Pat. No. 5,013,547 and WO 92/07466.

An overview of the prior art which is hereby incorporated into the present application is given:

for radioimmunotherapy by S. J. DeNardo et al. in “A new era for radiolabeled antibodies in cancer?”, Current Opinion in Immunology, 1999, vol. 11, pages 563-569,

for immunotoxins by R. J. Kreitman in “Immunotoxins in Cancer”, Current Opinion in Immunology, 1999, vol 0.11, pages 570-78,

for bispecific antibodies by D. M. Segal et al. in “Bispecific antibodies in cancer therapy”, Current Opinion in Immunology, 1999, vol. 11, page 558-562,

for the preparation of single-chain Fv-antibodies in U.S. Pat. No. 5,260,203,

for the preparation of antibody fragments conjugated or expressed as a single polypeptide chain in WO 91/19739,

for the preparation of humanized antibodies in U.S. Pat. No. 5,859,205,

for the preparation of chimeric antibodies (“CDR grafting”) in EP 0 620 276,

for the preparation of cross-linked antibodies in U.S. Pat. No. 5,714,149,

for the preparation of antibody-cytokine fusion proteins by S. D. Gillies et al, in “Antibody-targeted interleukin 2 stimulates T-cell killing of autologous tumor cells”, Proc. Natl. Acad. Sci U.S.A., 1992, vol. 89, pages 1428-1432 or by T. Dreier et al. in “Recombinant immunocytokines targeting the mouse transferrin receptor; construction and biological activities”, Bioconjug. Chem. 1998, vol. 9, pages 482-489.

The afore-mentioned antibodies can be used to identify plasma cells (preferably human plasma cells), and, for instance, to sort them out. For instance, all that is needed for this purpose is to label the antibodies of the invention with a fluorochrome in a conventional manner and to carry out fluorescence-activated cell sorting (FACS). Additional separation methods are known from the state of the art (see for instance Greiner A., et al. (1997) American Journal of Pathology, vol. 150, pages 1583-1593). However, further methods of immunoisolation and immunoseparation are known in the art and may comprise the binding to solid supports, like beads, preferably magnetic beads etc.

In this manner, it is possible to separate and isolate all plasma cells from a blood sample or tissue. In this process, the degenerated plasma cells are also removed, in order to eliminate the plasma cells causing the tumor or the autoimmune disease.

In particular, labeling and removal of the plasma cells can also be carried out outside the body, for instance in an outer artificial blood circulation in a manner similar to dialysis.

The invention also relates to an antigen, characterized in that it binds to and/or is recognized by the antibody of the invention.

The antigen of the invention is labeled by the antibody of the invention. One of these antigens is characterized in that it is a surface protein of plasma cells and possesses a molecular weight of about 94 kD or of about 55 kD. It is in particular preferred that the molecular weight of the antigen of the present invention is between 50 and 60 kD, most preferably around 50 to 55 kD. Said molecular weight can be deduced employing, inter alia, the following method: A cell suspension (107 cells) is biotinylated by addition of sulfo-NHS-biotin (MW 400; Pierce). Said cells, for example patient cells, may be lyzed in CHAPS-buffer and antibodies as disclosed herein, e.g. the antibody secreted by the hybridoma cell line DSM ACC 2441, may be employed in immunoprecipitation approaches. Immunoprecipitated material may be solubilized in SDS-sample buffer and the sample may be electrophoresed employing a 10% SDS-PAGE. As molecular weight standards, commercially available standards like HMW standards from SIGMA (not-prestained) may be employed. The electrophoresed proteins may be transferred onto a nitrocellular membrane and the immunoprecipitated, biotinylated proteins can be visualized employing Streptavidin-HRP (e.g. from Pharmacia) and ECL-systems. This antigen has been found to occur not only on the surface of the plasma cells but also in their cell plasma.

Therefore, the present invention also relates to antibodies which are characterized in that it binds the above described antigen and/or an epitope thereof. Said antibody may be obtained by conventional methods and/or methods described herein. In particular, said antibody may be obtained by immunizing an animal with the antigen of the invention and/or with an epitope thereof and by isolating the formed antibody from the blood of said animal (see, inter alia, Harlow and Lane, loc. cit.).

The antibody(ies) may also be prepared by using the cells of the immunized animal to produce a cell line which produces said antibody as a monoclonal antibody, in that this cell line is cultured and the antibody produced is isolated. Preferably said cultural cell is a cell derived from a spleen cell of the immunized animal, preferably said cell is a hybridoma cell prepared by fusion with a myeloma cell (see also appended examples).

In a preferred embodiment of the invention, the antibody of the invention is used for identifying and/or characterizing the corresponding antigen and/or for specifically labeling/detecting/recognizing plasma cells, preferably human plasma cells. Said antibody may also be employed to prepare additional antibodies, fragments or derivatives thereof which label/recognize plasma cells. The invention also relates to the nucleotide sequences/polynucleotides encoding at least one variable region of these antibodies, fragments or derivatives thereof.

It has to be stressed that also a cDNA expression library in E. coli can be screened indirectly for peptides having at least one epitope of the antigen of the invention using antibodies of the invention (Chang and Gottlieb, J. Neurosci., 8:2123, 1988). After having revealed the structure of such antigens the rational design of binding partners and/or domains may be possible. For example, folding simulations and computer redesign of structural motifs can be performed using appropriate computer programs (Olszewski, Proteins 25 (1996), 286-299; Hoffman, Comput. Appl. Biosci. 11 (1995), 675-679). Furthermore, computers can be used for the conformational and energetic analysis of detailed protein models (Monge, J. Mol. Biol. 247 (1995), 995-1012; Renouf, Adv. Exp. Med. Biol. 376 (1995), 37-45).

The knowledge of this antigen now allows this antigen to be purified in a simple conventional manner, for instance by gel electrophoresis, and an animal organism to be immunized in turn by this antigen, in order to produce additional antibodies against the same antigen. Of course, the thus produced antibodies then again label only plasma cells specifically, as the cells present the 94 kD-antigen or the 55 kD antigen on their surfaces, and can be used as described above.

Hence, a defined method for producing any additional antibodies specifically directed against plasma cells is available which can be carried out by standard immunological techniques, as described in many textbooks (for instance Janeway et al., Immunologie, 1995, Spektrum-Verlag Heidelberg, Berlin, Oxford). Of course, after immunization of the animal, the corresponding antibody can again be produced as a monoclonal antibody in a conventional manner, as known from the state of the art.

The present invention also provides for multifunctional polypeptides wherein at least one part of this polypeptide comprises a binding site of an immunoglobulin chain or of an antibody of this invention.

Preferably said multifunctional polypeptide is a single-chain multi-functional polypeptide comprising

(a) a first domain comprising a binding-site of an immunoglobulin chain or an antibody as defined herein; and

(b) a second domain comprising a binding site of an immunoglobulin chain or an antibody specifically recognizing the CD3 antigen.

The terms “first domain” and “second domain” in accordance with the present invention mean that one binding site is directed against an epitope of the antigen of the invention, e.g. an specific epitope of an antigen on plasma cells, preferably of human plasma cells, the other binding site is directed against the CD3 antigen of T cells, preferably human T cells.

The term “binding site” as used in accordance with the present invention denotes a domain comprising a three-dimensional structure capable of specifically binding to an epitope like native antibodies, free scFv fragments or one of their corresponding immunoglobulin chains, preferably the V_{H} chain. Thus, said domain can comprise the V_{H} and/or V_{L} domain of an antibody or an immunoglobulin chain, preferably at least the V_{H} domain. On the other hand, said binding sites contained in the polypeptide of the invention may comprise at least one complementarity determining region (CDR) of an antibody or immunoglobulin chain recognizing the antigen of the invention and CD3 antigens, respectively. In this respect, it is noted that the domains of the binding sites present in the polypeptide of the invention may not only be derived from antibodies but also from other binding proteins recognizing CD3 or the antigen of the invention, such as naturally occurring surface receptors or ligands. In accordance with the invention, said binding site is comprised in a domain.

The term “multifunctional polypeptide” as used herein denotes a polypeptide comprising at least two amino acid sequences derived from different origins, i.e. from two different molecules, optionally derived from different species wherein at least two of said origins specify the binding sites. Accordingly, said binding sites specify the functions or at least some functions of said multifunctional peptide. Such polypeptides include, for example, bispecific single-chain (bsc) antibodies.

The term “single-chain” as used in accordance with the present invention means that said first and second domain of the polypeptide are covalently linked, preferably in the form of a co-linear amino acid sequence encodable by a nucleic acid molecule.

CD3 denotes an antigen that is expressed on T-cells as part of the multimolecular T-cell receptor complex and that consists of three different chains CD3ε, CD3ε and CD3γ. Clustering of CD3 on T-cells, e.g., by immobilized anti-CD3-antibodies, leads to T-cell activation similar to the engagement of the T-cell receptor but independent from its clone typical specificity. Actually, most anti-CD3-antibodies recognize the CD3ε-chain.

Antibodies that specifically recognize the CD3 antigen are described in the prior art and can be generated by conventional methods known in the art, see, inter alia, WO 99/54440.

The single chain multifunctional polypeptide may be particularly useful in medical purposes, e.g. the treatment of tumors, in particular of lymphomas and/or plasmacytomas, multiple myelomas or in the treatment of immunological disorders, like autoimmune diesases.

In a preferred embodiment of the polypeptide of the invention said domains of the multifunctional polypeptide are connected by a polypeptide linker. Said linker is disposed between said first and said second domain, wherein said polypeptide linker preferably comprises plural, hydrophilic, peptide-bonded amino acids and connects the N-terminal end of said first domain and the C-terminal end of said second domain.

In a further preferred embodiment of the invention said first and/or second domain of the above-described polypeptide mimic or correspond to a V_{H} and V_{L} region from a natural antibody. The antibody providing the binding site for the polypeptide of the invention can be, e.g., a monoclonal antibody, polyclonal antibody, chimeric antibody, humanized antibody, bispecific antibody, synthetic antibody, antibody fragment, such as Fab, Fv or scFv fragments etc., or a chemically modified derivative of any of these as discussed herein above.

In a further preferred embodiment of the invention at least one of said domains in the above-described polypeptide is a single-chain fragment of the variable region of the antibody.

As is well known, Fv, the minimum antibody fragment which contains a complete antigen recognition and binding site, consists of a dimer of one heavy and one light chain variable domain (V_{H} and V_{L}) in non-covalent association. In this configuration that corresponds to the one found in native antibodies the three complementarity determining regions (CDRs) of each variable domain interact to define an antigen binding site on the surface of the V_{H}-V_{L} dimer. Collectively, the six CDRs confer antigen binding specificity to the antibody. Frameworks (FRs) flanking the CDRs have a tertiary structure which is essentially conserved in native immunoglobulins of species as diverse as human and mouse. These FRs serve to hold the CDRs in their appropriate orientation. The constant domains are not required for binding function, but may aid in stabilizing V_{H}-V_{L} interaction. Even a single variable domain (or half of an Fv comprising only three CDRs specific for an antigen) has the ability to recognize and bind antigen, although usually at a lower affinity than an entire binding site (Painter, Biochem. 11 (1972), 1327-1337). Hence, said domain of the binding site of the polypeptide of the invention can be a pair of V_{H}-V_{L}, V_{H}-V_{H} or V_{L}-V_{L} domains either of the same or of different immunoglobulins. The order of V_{H} and V_{L} domains within the polypeptide chain is not decisive for the present invention, the order of domains given hereinabove may be reversed usually without any loss of function. It is important, however, that the V_{H} and V_{L} domains are arranged so that the antigen binding site can properly fold. The preparation of multifunctional polypeptides is well known in the art, as, inter alia, disclosed in WO 99/54440 or WO 00/06605.

In a preferred embodiment of the polypeptides of the invention said domains are arranged in the order V_{L}WUE-1-V_{H}WUE-1-V_{H}CD3-V_{L}CD3, wherein “V_{L}” and “V_{H}” means the light and heavy chain of the variable domain of specific anti-WUE-1 and anti-CD3 antibodies.

As discussed above, said binding sites are preferably connected by a flexible linker, preferably by a polypeptide linker disposed between said domains, wherein said polypeptide linker comprises plural, hydrophilic, peptide-bonded amino acids of a length sufficient to span the distance between the C-terminal end of one of said domains comprising said binding sites and the N-terminal end of the other of said domains comprising said binding sites when the polypeptide of the invention assumes a conformation suitable for binding when disposed in aqueous solution.

Preferably, said polypeptide linker comprises a plurality of glycine, alanine and/or serine residues. It is further preferred that said polypeptide linker comprises a plurality of consecutive copies of an amino acid sequence. Usually, the polypeptide linker comprises 1 to 15 amino acids although polypeptide linkers of more than 15 amino acids may work as well. In a preferred embodiment of the invention said polypeptide linker comprises 1 to 5 amino acid residues.

In a particularly preferred embodiment of the present invention said polypeptide linker in the polypeptide of the invention comprises 5 amino acids. As demonstrated in the appended examples, said polypeptide linker advantageously comprises the amino acid sequence Gly Gly Gly Gly Ser.

In a most preferred embodiment of the present invention, the multifunctional polypeptide as defined herein above is a single-chain antibody. It is particularly preferred that the multifunctional polypeptide is encoded by the nucleic acid molecule as depicted in the appended examples and in SEQ ID NO: 21, preferably comprising the amino acid sequence as shown in SEQ ID NO: 22.

The present invention further relates to a (multifunctional) polypeptide comprising at least one further domain, said domains being linked by covalent or non-covalent bonds. Said multifunctional polypeptide may, therefore, be in the format of an “heterominibody” as discussed in WO 00/06605.

The linkage can be based on genetic fusion according to the methods known in the art and described above or can be performed by, e.g., chemical cross-linking as described in, e.g., WO 94/04686. The additional domain present in the polypeptide of the invention may preferably be linked by a flexible linker, advantageously a polypeptide linker to one of the binding site domains wherein said polypeptide linker comprises plural, hydrophilic, peptide-bonded amino acids of a length sufficient to span the distance between the C-terminal end of one of said domains and the N-terminal end of the other of said domains when said polypeptide assumes a conformation suitable for binding when disposed in aqueous solution. Preferably, said polypeptide linker is a polypeptide linker as described in the embodiments hereinbefore. The polypeptide of the invention may further comprise a cleavable linker or cleavage site for proteinases, such as enterokinase; see also the appended examples.

Furthermore, said additional domain may be of a predefined specificity or function. For example, the literature contains a host of references to the concept of targeting bioactive substances such as drugs, toxins, and enzymes to specific points in the body to destroy or locate malignant cells or to induce a localized drug or enzymatic effect. It has been proposed to achieve this effect by conjugating the bioactive substance to monoclonal antibodies (see, e.g., N.Y. Oxford University Press; and Ghose, J. Natl. Cancer Inst. 61 (1978), 657-676).

In this context, it is also understood that the polypeptides according to the invention may be further modified by conventional methods known in the art. This allows for the construction of chimeric proteins comprising the polypeptide of the invention and other functional amino acid sequences, e.g., nuclear localization signals, transactivating domains, DNA-binding domains, hormone-binding domains, protein tags (GST, GFP, h-myc peptide, FLAG, HA peptide) which may be derived from heterologous proteins.

In context of the present invention, the invention also relates to polynucleotides/nucleic acid molecules encoding the multifunctional polypeptides of the invention comprised in vectors and/or transfected into host cells as described herein above. In general, the term “polynucleotide”, “nucleic acid molecules” and “nucleotide sequences” relate to DNA, RNA, PNA as well as recombinantly produced chimeric nucleic acid molecules comprising any of these polynucleotides either alone or in combination.

In a further embodiment the present invention relates to a composition comprising the antibodies or functional fragments or derivatives thereof, the multifunctional polypeptide, the nucleotide sequences/polynucleotides, vectors and/or cells of the invention. Preferably said composition is a pharmaceutical or a diagnostic composition.

The pharmaceutical composition of the present invention may further comprise a pharmaceutically acceptable carrier. Examples of suitable pharmaceutical carriers are well known in the art and include phosphate buffered saline solutions, water, emulsions, such as oil/water emulsions, various types of wetting agents, sterile solutions, etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intramuscular, topical or intradermal administration. The dosage regiment will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. However, a more preferred dosage for continuous infusion might be in the range of 0.01 μg to 10 mg units per kilogram of body weight per hour. Particularly preferred dosages are recited herein below. Progress can be monitored by periodic assessment. Dosages will vary but a preferred dosage for intravenous administration of DNA is from approximately 10⁶ to 10¹² copies of the DNA molecule. The compositions of the invention may be administered locally or systematically. Administration will generally be parenterally, e.g., intravenously; DNA may also be administered directed to the target site, e.g., by biolistic delivery to an internal or external target site or by catheter to a site in an artery. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishes, electrolyte replenishers (such as those based on Ringer's dextrose), and the like. Preservatives and other additives may also be present such as, for example, antimicrobials, anti-oxidants, chelating agents, and inert gases and the like. In addition, the pharmaceutical composition of the present invention might comprise proteinaceous carriers, like, e.g., serum albumine or immunoglobuline, preferably of human origin. Furthermore, it is envisaged that the pharmaceutical composition of the invention might comprise further biologically active agents, depending on the intended use of the pharmaceutical composition. Such agents might be drugs acting on the gastrointestinal system, drugs acting as cytostatica, or drugs employed in the treatment of autoimmune diseases, (multiple) lymphomas, multiple myelomas or plasma cytomas.

It is envisaged by the present invention that the various polynucleotides and vectors of the invention are administered either alone or in any combination using standard vectors and/or gene delivery systems, and optionally together with a pharmaceutically acceptable carrier or excipient. Subsequent to administration, said polynucleotides or vectors may be stably integrated into the genome of the subject.

On the other hand, viral vectors may be used which are specific for certain cells or tissues and persist in said cells. Suitable pharmaceutical carriers and excipients are well known in the art. The pharmaceutical compositions prepared according to the invention can be used for the prevention or treatment or delaying of different kinds of diseases, which are related to malignancies, in particular lymphomas, multiple myelomas, plasma-cytomas and autoimmune diseases.

Furthermore, it is possible to use a pharmaceutical composition of the invention which comprises polynucleotide or vector of the invention in gene therapy. Suitable gene delivery systems may include liposomes, receptor-mediated delivery systems, naked DNA, and viral vectors such as herpes viruses, retroviruses, adenoviruses, and adeno-associated viruses, among others. Delivery of nucleic acids to a specific site in the body for gene therapy may also be accomplished using a biolistic delivery system, such as that described by Williams (Proc. Natl. Acad. Sci. USA 88 (1991), 2726-2729). Further methods for the delivery of nucleic acids comprise particle-mediated gene transfer as, e.g., described in Verma, Gene Ther.15 (1998), 692-699.

It is to be understood that the introduced polynucleotides and vectors express the gene product after introduction into said cell and preferably remain in this status during the lifetime of said cell. For example, cell lines which stably express the polynucleotide under the control of appropriate regulatory sequences may be engineered according to methods well known to those skilled in the art. Rather than using expression vectors which contain viral origins of replication, host cells can be transformed with the polynucleotide of the invention and a selectable marker, either on the same or separate plasmids. Following the introduction of foreign DNA, engineered cells may be allowed to grow for 1-2 days in an enriched media, and then are switched to a selective media. The selectable marker in the recombinant plasmid confers resistance to the selection and allows for the selection of cells having stably integrated the plasmid into their chromosomes and grow to form foci which in turn can be cloned and expanded into cell lines. Such engineered cell lines are also particularly useful in screening methods for the detection of compounds involved in, e.g., B-cell/T-cell interaction.

A number of selection systems may be used, including but not limited to, the herpes simplex virus thymidine kinase (Wigler, Cell 11 (1977), 223), hypoxanthine-guanine phosphoribosyltransferase (Szybalska, Proc. Natl. Acad. Sci. USA 48 (1962), 2026), and adenine phosphoribosyltransferase (Lowy, Cell 22 (1980), 817) in tk⁻, hgprt⁻ or aprt⁻ cells, respectively. Also, antimetabolite resistance can be used as the basis of selection for dhfr, which confers resistance to methotrexate (Wigler, Proc. Natl. Acad. Sci. USA 77 (1980), 3567; O'Hare, Proc. Natl. Acad. Sci. USA 78 (1981), 1527), gpt, which confers resistance to mycophenolic acid (Mulligan, Proc. Natl. Acad. Sci. USA 78 (1981), 2072); neo, which confers resistance to the aminoglycoside G-418 (Colberre-Garapin, J. Mol. Biol. 150 (1981), 1); hygro, which confers resistance to hygromycin (Santerre, Gene 30 (1984), 147); or puromycin (pat, puromycin N-acetyl transferase). Additional selectable genes have been described, for example, trpB, which allows cells to utilize indole in place of tryptophan, hisD, which allows cells to utilize histinol in place of histidine (Hartman, Proc. Natl. Acad. Sci. USA 85 (1988), 8047); and ODC (ornithine decarboxylase) which confers resistance to the ornithine decarboxylase inhibitor, 2-(difluoromethyl)-DL-ornithine, DFMO (McCologue, 1987, In: Current Communications in Molecular Biology, Cold Spring Harbor Laboratory ed.).

The compounds of the present invention, in particular the antibodies may be particularly useful in filtering systems. For example, when therapeutically desired the antibodies or functional fragments or derivatives thereof of the invention may be employed in so-called “purging”-strategies, wherein the blood of or bone marrow of a patient, e.g. a plasmacytoma patient, is freed from its malignant plasma cells, e.g. its tumor cells during bone marrow transplantation.

The diagnostic composition of the invention comprising any one of the above described antibodies, (multifunctional) polypeptides, polynucleotides, vectors or (host) cells of the invention may optionally comprise suitable means for detection.

The antibodies as well as the (multifunctional) polypeptides of the invention are also suited for use in immunoassays in which they can be utilized in liquid phase or bound to a solid phase carrier. Examples of immunoassays which can utilize the polypeptide of the invention are competitive and non-competitive immunoassays in either a direct or indirect format. Examples of such immunoassays are the radioimmunoassay (RIA), the sandwich (immunometric assay) and the Western blot assay. The antibodies as well as polypeptdies are also useful in immuno-isolation approaches as well as in immuno-precipitation.

The antibodies as well as polypeptides of the invention can be bound to many different carriers and used to isolate cells specifically bound to said antibodies as well as polypeptides. Examples of well-known carriers include glass, polystyrene, polyvinyl chloride, polypropylene, polyethylene, polycarbonate, dextran, nylon, amyloses, natural and modified celluloses, colloidal metals, polyacrylamides, agaroses, and magnetite. The nature of the carrier can be either soluble or insoluble for the purposes of the invention.

There are many different labels and methods of labeling known to those of ordinary skill in the art. Examples of the types of labels which can be used in the present invention include enzymes, radioisotopes, colloidal metals, fluorescent compounds, chemiluminescent compounds, and bioluminescent compounds; see also the embodiments discussed hereinabove. The diagnostic composition of the present invention is particular useful in detecting, labeling as well as isolating plasma cells, preferably human plasma cells. The diagnostic composition is also useful for the in vivo, and in vitro study of B-cell lymphomas, the mechanism of B-cell differentiation, or the biology of plasma cells.

The present invention also relates to the use of the compounds of the invention described hereinabove for the preparation of a pharmaceutical composition for the treatment of plasma cell malignancies, in particular multiple myelomas, lymphomas, plasmacytomas (tumors) as well as autoimmune diseases.

The present invention also provides for a kit comprising the antibody or a functional fragment or a derivative thereof of the present invention, the antigen of the invention, the epitope of the invention, the (multifunctional) polypeptide of the invention, the nucleotide sequence of the invention and/or the vector and/or host of the invention.

Such a kit may comprise a carrier means being compartmentalized to receive in close confinement one or more container means such as vials, tubes and the like, each of the container means comprising one of the separate elements to be used in the method. For example, one of the container means may comprise an antigen or epitope of the invention bound to a carrier. A second container may comprise soluble, detectably-labeled second antibody, in lyophilized form or in solution. In addition, the carrier means may also contain a plurality of containers each of which comprises different, predetermined amounts of the antigen or epitopes of the invention.

In summary, regarding plasma cells, only specific antibodies directed against cytoplasmic components have so far been available according to the state of the art, and hence were incapable of recognizing the plasma cell surface. The antibodies of the invention are, for the first time, directed against antigens which are expressed on the cell surface of plasma cells and are specific for plasma cells. Hence, for the first time, antibodies are available, which can be used in the diagnosis and therapy of, for instance, autoimmune diseases, lymphomas or plasmocytomas, and offer a substitute for chemotherapy with all its side effects, which was the only therapy so far known in the art.

The figures show:

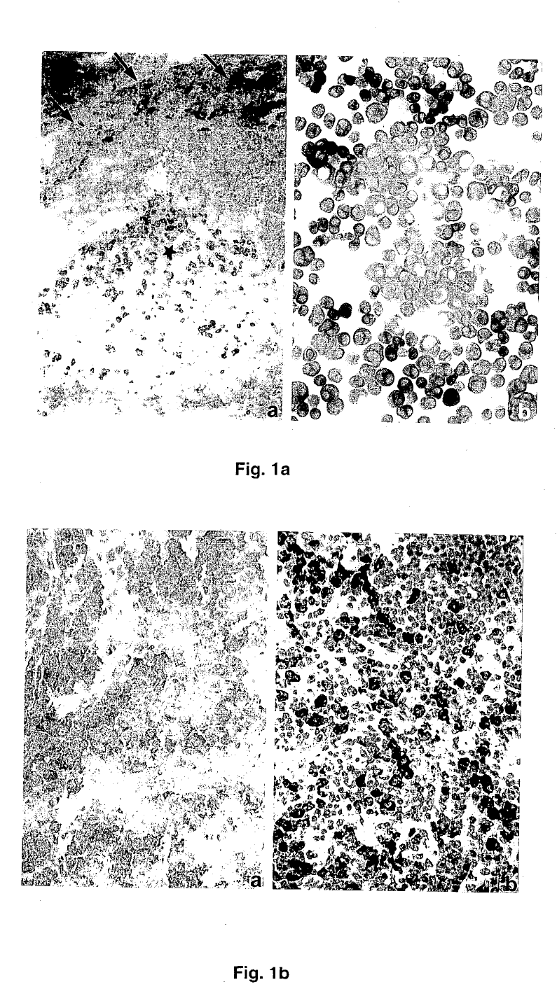

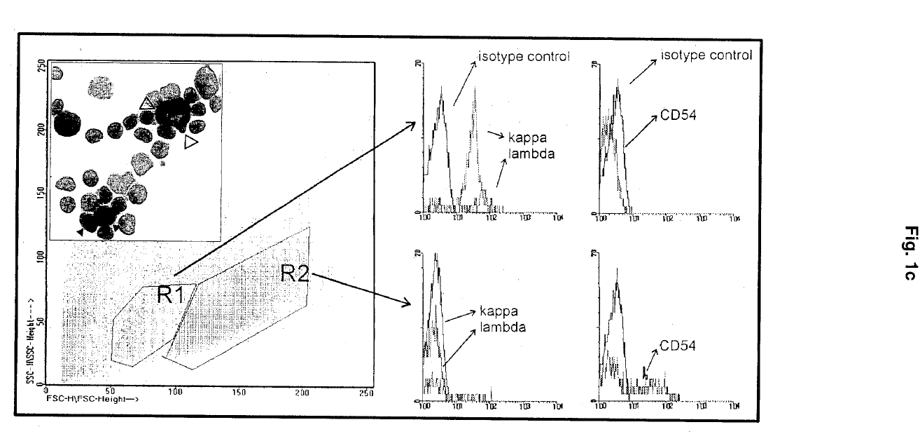

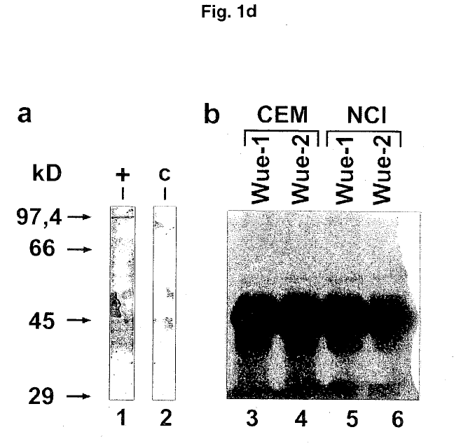

FIG. 1 Immunohistochemistry employing the antibody of the invention.

FIG. 1a Secondary lymphatic tissue with a germinal center in the middle (star) and individual brown-stained plasma cells in the upper third of this germinal center.

Partial picture b of FIG. 1a shows immunostaining of the plasma cell line NCI-923, in which all cells are immunohistochemically strongly positively labeled with Wue-1.

FIG. 1b In partial picture a of FIG. 1b, a B-cell tumor (a so-called little malignant MALT lymphoma) differentiating into plasma cells can be seen with a diffuse host of positively labeled plasma cells apart from the not yet differentiated tumor part of the lymphoma which produces a Wue-1 negative reaction. In partial picture b of FIG. 1b, a highly-malignant lymphoma is labeled with Wue-1 which corresponds to malignant plasma cells (so-called anaplastic plasmocytoma) and has a cytoplasma staining and membrane staining.

FIG. 1c Picture insert: immunohistochemistry of mature plasma cells. FACS analysis.

FIG. 1d Western blot, in which a plasma cell line (NC1-H929) served as an antigen source.

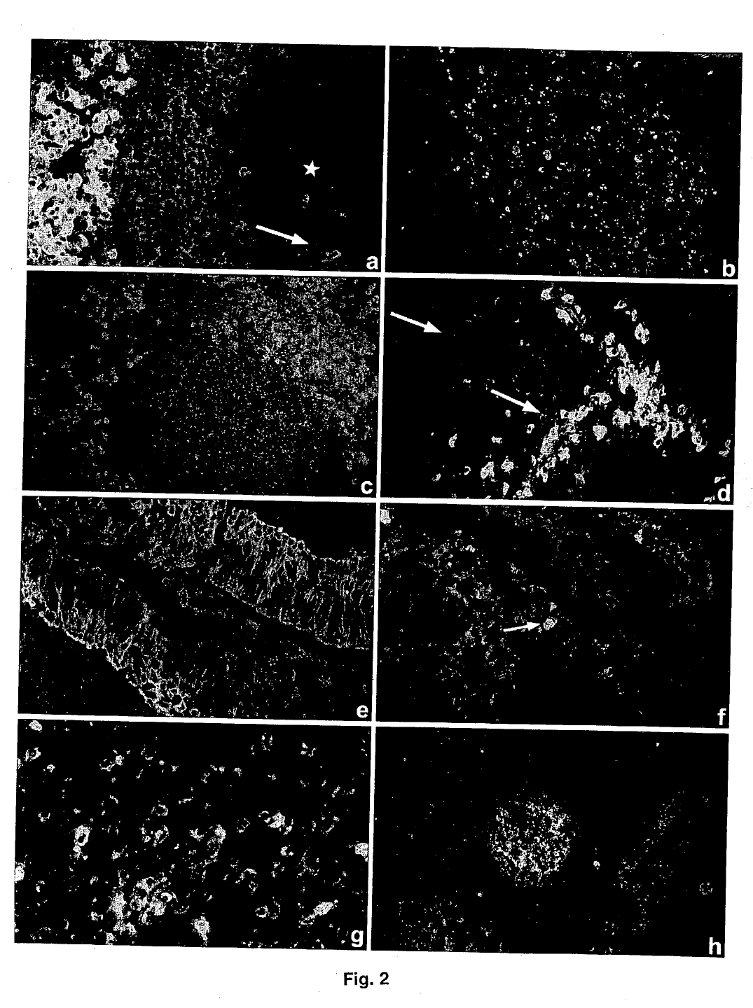

FIG. 2 In all partial pictures a to h of FIG. 2, the specific binding pattern which is selective for plasma cells is described on the basis of human tonsils.

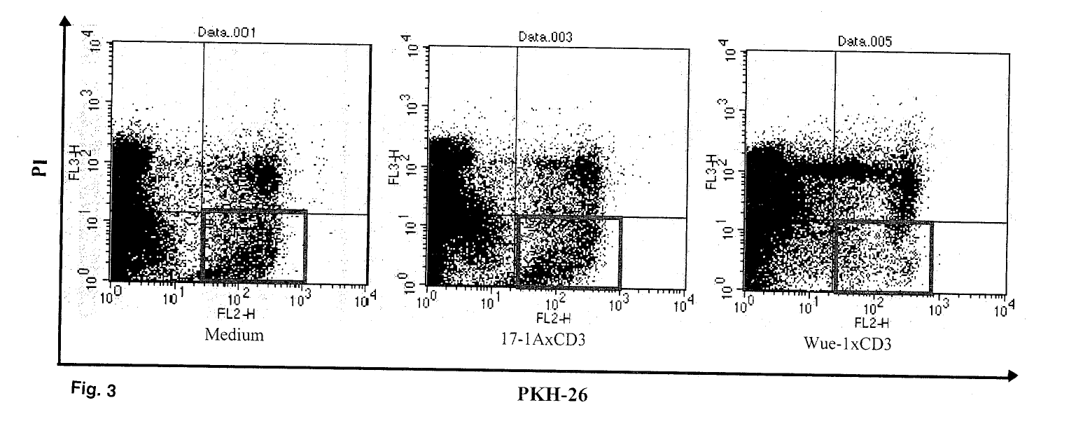

FIG. 3 Cytotoxicity assay performed with MM cells enriched by CD138-based immunoisolation. The fraction of living myeloma cells was positive for the PKH-26 dye (measured in FL-2) and negative for PI (measured in FL-3). Dead MM cells were positive for both, PKH-26 and PI. PBMCs were negative for PKH-26. A strong loss of PI-negative myeloma cells is detectable in the sample containing bscWue-1xCD3 compared to bscl7-1AxCD3 after an incubation time of 4 days and an E:T ratio of 10:1. Samples were analyzed as triplicates showing a mean specific lysis of 56% of target cells by autologous prestimulated PBMCs.

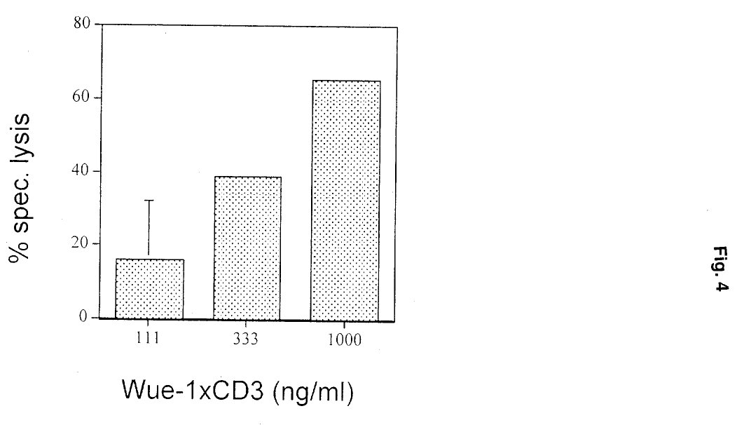

FIG. 4 Dose dependent activity of bscWue-1xCD3. Assay incubation time was 6 days and the E:T ratio 10:1. Samples were analyzed as duplicates showing a maximium specific lysis of 65% of target cells by autologous prestimulated PBMCs.

In the following, an example of a method for preparing an antibody according to the invention and an example of an antibody according to the invention is given. Furthermore, the preparation of a single-chain multifunctional compound and its corresponding use in accordance with the invention is illustrated.

### EXAMPLE 1

### The method for preparing an antibody (“Wue-1”)

a) Immunization of a mouse

BALB/c—mice were immunized with cells of a cell line which was obtained from a lymphoma of the MALT type (cell line No. H3302/88, Dr. A. Greiner, Institut für Pa-thologie, Universitat Würzburg). These cells are capable of differentiating into plasma cells in vitro.

b) Isolation of Spleen Cells of the Immunized Mouse

The sterilely removed spleen was placed into a petri dish containing 5 ml of RPMI without serum and pressed through a nylon gauze net (pore size 100 μm). Subsequently, the cell suspension was resuspended several times with a 5 ml pipette and transferred to a 50 ml vial. The thus obtained spleen cells were washed twice with 20 ml of HBSS. That is to say, the cells were centrifuged at 1500 rpm for 5 minutes in each case, the supernatant was removed and the pellet was resuspended in 20 ml of HBSS in each case.

c) Preparation of the Fusion Partner (Myeloma Cells)

The myeloma cells (P3x63 Ag8.653, see Kearney J. F. et al. (1979) Journal of Immunology, vol. 123, page 1548-1550) were kept in azaguanin medium (1 mg/ml RPMI+10% FKS) for some days prior to fusion. For the fusion of a mouse spleen about 4×10⁷ myeloma cells were needed (this is the content of about one vial of a confluently grown cell culture of 175 cm²). The myeloma cells were removed from the bottom of the vial by means of EDTA buffer, and subsequently washed with HBSS, like the spleen cells, and again centrifuged, and the supernatant above the pellet was sucked off.

d) Cell Fusion

The suspension with spleen cells from step 2 was placed on the pellet from myeloma cells from step 3 and mixed with it.

Fusion was carried out at +37° C. according to standard procedures (see for instance Greiner et al. (1994) “Monoclonal gammapathies III. Clinical significance and basic mechanism” (editors Radl et al.), pages 187-190, EURAGE, Leiden, the Netherlands). To this end, all required media were preheated to +37° C. in a water bath.

Within one minute after mixing the spleen cells with the myeloma cells, 1 ml of 50% PEG was slowly dropped into the cell mixture while the mixture was stirred, in order to prevent lump formation. After waiting for 1.5 minutes, first 15 ml RPMI without serum and then 20 ml RPMI+10% FKS were slowly dropped into the mixture, while the mixture was stirred. The cell mixture of myeloma fusion partner cells and mouse spleen cells was centrifuged at 1500 rpm for 5 minutes, the supernatant was removed and the pellet resuspended in 125 ml of RPMI+10% FKS+200 units per milliliter IL-6+0.5 ml per 100 ml of gentamycin. The cell suspension thus obtained was distributed in milliliters into five 24-well plates. After culturing for about 24 hours, 1 ml of RPMI+10% FKS+200 units per milliliter IL-6+0.5 ml per 100 ml of gentamycin+2×HAT was added per well.

e) Culturing of Hybridoma Cells

The fused hybridoma cells were supplied with new medium (RPMI+10% FKS+200 units per milliliter IL-6+0.5 ml per 100 ml gentamycin+1×HAT) every 4 days. For this purpose, the culture supernatant of the wells was completely sucked off (hybridoma cells grow adherently), and 2 ml of new medium were added per well. After 14 days, the ‘HAT medium’ was exchanged for ‘HT-medium’ (RPMI+10% FKS+200 units per milliliter IL-6+0.5 ml per 100 ml gentamycin+1% HAT). If growing clones covered the field of view in the microscope, they were tested for immunoglobulin production. All positive clones were transferred into small culture vials (25 cm²).

f) Specificity Test

All positive clones were immuno-histochemically tested for specificity by being cut in the frozen state and subjected to immunostaining (see below).

After repeated recloning, a stable cell line was obtained.

g) Isolation of the Antibodies Produced.

The supernatant of the cultures of the afore-mentioned cell line was removed and purified by precipitation with ammonium sulfate and subsequent protein A affinity chromatography and labeled with FITC or biotin using N-hydroxy-succinimide ester (company Sigma, Germany) in accordance with standard methods (Greiner et al (1994) Lab. Invest., vol. 70, pages 572-578). The antibody produced by this cell line is hereinafter referred to as “Wue-1”. It was identified by isotype IgG2a.

h) The antibody Wue-1 was characterized in respect of its amino acid sequence. The variable region Fw(L) of its light chains (L-chains) has the following sequence

Its partial sequence
DIVMTQTPLTLSVTIGQPASLSC corresponds to variable region FW-1
KSSQSLLDSDGKTYLN corresponds to the complementarity-determining region
CDR-1
WLLQRPGQSPKRLIS corresponds to variable region FW-2
LVSKLDS corresponds to the complementarity-determining region CDR-2
GVPDRFTGSGSGTDFTLKISRVEAEDLGVYYC corresponds to variable
region FW-3
WQGTHLPWT corresponds to the complementarity-determining region GDR-
3
and
FGGGTKLEIKR corresponds to variable region FW-4.

The variable region Fw(H) of its heavy chains (H-chains) shows the following sequence:

QVQLQQSGPELVKTGASVKISCKGSGYSFSGYYMHWVKQSHGKRLEWIG
YISGYNGDTRYNQKFRGKATFIVDISSRTAYMQFNSLTSEDSAVYYCARGG
YYGYVDYWGQGTTLTVSS
Its partial sequence
QVQLQQSGPELVKTGASVKISCKGSGYSFS corresponds to var-
iable region
FW-1,
GYYMH corresponds to the complementarity-determin-
ing region CDR-1,
WVKQSHGKRLEWIG corresponds to variable region FW-2,
YISGYNGDTRYNQKFRG corresponds to the complementar-
ity-determining
region CDR-2
KATFIVDISSRTAYMQFNSLTSEDSAVYYCAR corresponds to
variable
region FW-3,
GGYYGYVDY corresponds to the complementarity-deter-
mining region CDR-
3
and
WGQGTTLTVSS correspond to variable region FW-4.

Moreover, the nucleotide sequence of gene sequences encoding antibody Wue-1 was analyzed. The gene coding for the light L-chains of the antibody Wue-1 contains the following nucleotide sequence:

(SEQ ID NO: 15)
gaagcacgcg tagatatcgt gatgacccaa actccactca ctttgtcggt taccattgga
caaccagcct ccctctcttg caagtcaagt cagagcctct tagatahtga tggaaagaca tatttgaatt
ggttgttaca gaggccaggc cagtctccaa agcgcctaat ctctctggtg tctaaattgg actctggagt
ccctgacaga ttcactggca gtggatcagg gacagatttc acactgaaaa tcagcagagt
ggaggctgag gatttgggag tctattattg ctggcaaggt acacatcttc cgtggacatt
cggtggaggc accaagctgg aaatcaaacg ggctgatgct gcggccgctg gatccatctt c

The gene coding for the heavy H-chains of antibody Wue-1 contains the following nucleotide sequence:

(SEQ ID NO: 16)
cgccatggcc gcgggattcc ggccatggcg caggtgcagc tgcagcagtc tggacctgag
ctagtgaaga ctggggcttc agtgaagata tcttgtaagg gttctggtta ctcattcagt ggttactaca
tgcactgggt caagcagagc catggaaaga ggcttgagtg gattggatat attagtggtt
ataatggtga tactaggtat aatcagaagt tcaggggcaa ggccacattt attgtagaca
tatcctccag gacagcctac atgcagttca acagcctgac atctgaagac tctgcggtct
attactgtgc aagagggggt tactacggct acgtggacta ctggggccaa ggcaccaccc
tcacagtctc ctcagccaaa acgacaccca agcttgtcta tccactggcc cctggtaatc
actgtgcggc cgccg

i) The above-mentioned materials used are defined as follows:

Nylon gauze of the company NORAS

8-azaguanin of the company Sigma No. Al 007

HBSS (Hank's salts) of the company Linaris No. F1431 KG

EDTA buffer of the company Linaris No. L1253 GG

PEG (polyethylene glycol 1500) of the company Boehringer

FKS (fetal calf serum) of the company Linaris No. S3181 KG

RPM! of the company Linaris No. F2613 KG

HAT supplement 50× of the company Linaris No. K1931 GG

HT supplement 50× of the company Linaris No. K1932 GG

Gentamycin of the company Linaris No. Fl 141 FG

IL-6 (interleukin-6) of the company Pharmingen, Hamburg

For the specificity tests, the antibody was purified from the cell culture supernatant by ammonium sulfate precipitation and subsequent affinity chromatography. The specificity test was carried out with preparations of fresh tissue cuts, cell centrifugation products and cell suspension solutions by immuno histochemistry.

j) Results:

Wue-1-positve cells were unambiguously and reproducibly identified by immunohistochemistry within secondary germinal centers and in the marginal zone and below the crypts of the tonsils (FIGS. 1A and 1B). The morphology and immunohistochemistry corresponds to mature plasma cells of either the Marschalko type or lymphoplasmacytoid type (FIG. 1C with picture insert).

In partial picture a of FIG. 1A a secondary lymphatic tissue with a germinal center in the middle (star) and individual brown-stained plasma cells in the upper third of this germinal center can be seen. This is the typical maturation zone for lymphocytes into plasma cells. Moreover, outside the follicles too, some positively labeled cells (arrows) can be seen near the surface epithelium at the upper margin of the picture. Partial picture b of FIG. 1A shows immunostaining of the plasma cell line NCI-923, in which all cells are immunohistochemically strongly positively labeled with Wue-1. The labeling pattern is in each case cytoplasmic and appears at the membrane, as has been shown by the non-permeabilizing FACS staining depicted in FIG. 1C.

Wue-1 did not react with macrophages or other lymphatic cells, such as T-lymphocytes or macrophages, and did not show any binding in the peripheral blood, liver tissue, kidneys, muscles, skin, urinary bladder, testis and ovary, and reacted weakly and inconstantly with secretory epithelium in the gastro intestinal duct (stomach, intestine).

Wue-1 did, however, react with all malignant tumors derived from plasma cells. By contrast, normal T- and B-lymphocytes and tumors from the early B-cell differentiation phase reacted negatively (FIG. 1B). In partial picture a of FIG. 1B, a B-cell tumor (a so-called little malignant MALT lymphoma) differentiating into plasma cells can be seen with a diffuse host of positively labeled plasma cells apart from the not yet differentiated tumor part of the lymphoma which produces a Wue-1 negative reaction. In partial picture b of FIG. 1B, a highly-malignant lymphoma is labeled with Wue-1 which corresponds to malignant plasma cells (so-called anaplastic plasmocytoma) and has a cytoplasma staining and membrane staining.

FIG. 1C, left-hand half, shows a FACS graph of a normal tonsil lymphocyte cell suspension. The cell size is in each case plotted on the x-axis, the cell granularity on the y-axis. R1 and R2 denote two cell populations, and according to general experience, the lymphocytes at rest are in R1, while the activated lymphocytes are in R2. An idea of this is conveyed by the inserted inner picture of the centrifuged cell suspension preparation which was stained with Wue-1. In this picture, small positive cells which correspond to the lymphoplasmacytoid plasma cells (arrow) and occur in R1 as well as large plasma cells of the Marschalko type from R2 can be seen.

In the right-hand half of FIG. 1C, four histograms can be seen, where the staining intensity is plotted on the x-axis and the cell number on the y-axis. The upper two histograms show the Wue-1 positive cells from R1. On the left-hand side top/bottom, the cells were additionally stained against light chain kappa and lambda antigens, on the right hand side top/bottom against the surface antigen CD54. A staining against an irrelevant antigen was superimposed on the pictures as a so-called isotype control, in order to show the extent of unspecific background staining. It is striking that lymphoplasmocytic cells (upper row) differ from mature plasma cells (lower row) in respect of the expression of light chains and CD54, as mature plasma cells do not express either one compared to the isotype control.

FIG. 1D shows a Western blot, in which a plasma cell line (NC1-H929) served as an antigen source. A positive band resulted at about 94 kD (FIG. 1D), that is to say the antigen labeled by Wue-1 has an apparent molecular weight of 94 kD.

Legend of FIG. 2 (Videoprint)

In all partial pictures a to h of FIG. 2, the specific binding pattern which is selective for plasma cells is described on the basis of human tonsils. A Cy3-fluorochrome conjugated Wue-1 mAB (red staining) with an optional choice of other defined antigens (green straining) is used as assay technique with double immunofluorescence, wherein an overlapping staining signal then appears yellow (color sum of green and red). Immunostaining was carried out according to current and generally used methods (for details see the relevant literature such as Janeway, Immunologie, Spektrum-Verlag, 1995). In summary, staining is first carried out indirectly with the unconjugated antibody (here: the antigens shown in green) and this antibody is then represented with a second secondary antibody labeled with green fluorescence. In a next step, all free valences of the secondary antibody are blocked with mouse serum, in order that Wue-1 should not be able to bind thereto unspecifically. Finally, staining is carried out with the second specificity (here: red-labeled Wue-1). Several specificity and sensitivity controls are conducted in parallel to each batch and indicate the dyeing behavior of the tissue while omitting the corresponding individual steps.

FIG. 2a shows double staining of Wue-1 (red) with CD44 (green). The germinal center of a lymphatic follicle can be seen in the illustration only vaguely and is therefore identified by a star. CD 44 labels mature B-cells (near the germinal center) and plasma cells (remote from the germinal center), with only the overlapping spectrum of the population remote from the germinal center being striking in double staining. An arrow marks individual plasma cells which are located in the germinal center but are typically CD44-negative.

In FIG. 2b, this finding is again especially emphasized. Here, a germinal center was selectively depicted. The cells which are outside the G0 phase (represented by Ki-67-positivity) and therefore proliferate, which is characteristic of germinal centers in lymphatic tissue, are labeled in green. Wue-1 positive cells are differentiated and no longer proliferate. Consequently, no double staining can be seen.

In FIG. 2c, a delimitation from another important cell population in the lymphatic tissue was carried out by means of CD3 staining. Consequently, in the tonsil, Wue-1 cells border on the T-cell outer zone (typical localization for plasma cells) but are negative for the Pan-T-cell marker.

In FIG. 2d, double staining with a known plasma cell antibody VS-38 which, however, only recognizes cytoplasmic antigens was carried out. Hence, there is high overlap and almost complete yellow staining. Moreover, individual cells are recognized by VS-38 and could be described as T-cells, which is a fact showing that VS-38 has a lower specificity for Wue-1.

FIG. 2e shows a staining which marks the tonsil surface in green (epithelium marker cytokeratin 8). This also shows that Wue-1 does not possess any cross reactivity, not even in the case of cells which characteristically migrate into the epithelium.

In FIG. 2f, co-expression with a functionally important antigen CD95 (FAS) was examined. Data regarding plasma cell expression have not been published so far.

In FIG. 2g, a combination of Wue-1 and an antibody 4D12 which recognizes the marginal zone B-cells, that is to say those cells which represent the direct precursors of plasma cells, was chosen. Consequently, double-labeled cells, which characterize transition, can also be seen apart from the corresponding individual staining.

In FIG. 2h, by contrast, double staining with CD23 was carried out, which appears on germinal center cells and immature B cells (so-called peripheral zone cells). The germinal center structures and peripheral zone structures of a lymphatic follicle are marked in green and there is a strip of unlabeled cells (T cell zone see FIG. 2c and marginal zone see FIG. 2g) and a Wue-1 positive outer zone.

The fact that Wue-1 specifically recognizes plasma cells has been shown in numerous tests both morphologically and immunohistochemically by means of multiple stainings (FIGS. 1A and 2 (videoprint)). In particular, the specificity and property of Wue-1 of binding to the membrane surface of normal and malignant plasma cells makes Wue-1 a valuable immunological tool, because Wue-1 can conversely also be coupled to carrier or messenger substances and can then bring diagnostic or therapeutic substances to the plasma cell both in vitro and in vivo, which is ensured by its ability to bind to the cell membrane of the plasma cells. Moreover, Wue-1 can be used therapeutically as a filter outside the body (so-called purging), in which the blood or bone marrow of a plasmacytoma patient is freed from its tumor cells during a bone marrow transplantation.

### EXAMPLE 2

### Wue-1 is Highly Specific for Healthy and Malignant Plasma Cells

The first step towards an antibody-based treatment of plasma cell malignancies (plasmacytoma/multiple myeloma) was to establish a monoclonal antibody specifically directed to plasma cells. Such an antibody was described above, Wue-1. A further characterization of Wue-1 was performed to determine the binding characterisctics of Wue-1 to lymphatic tissue. Human tonsil tissue was chosen for double-stainings with Wue-1 and a set of tissue markers. Wue-1 (DSM ACC2441) was used as described in the examples above.

Briefly, immunohistochemical stainings were performed on 4 micrometer cryostat sections of fresh frozen surgical specimen. Special attention was given to those organs known to be populated by plasma cells, i.e. bone marrow, lymph nodes, thymuses and tonsils. Furthermore, cytospin preparations of normal peripheral blood lymphocytes, secondary lymphoid tissues and lymphoma cell suspensions, and myeloma/plasmocytoma cell lines were subjected to immunocyto-chemical testing. For double staining on cryostat sections bound biotin-labeled antibodies were detected using Cy3-streptavidin (Dianova, Germany) as described (Greiner (1994) Lab Invest 70:572-578). The immunoperoxidase method was applied using a three-step incubation procedure with diluted purified monoclonal antibody Wue-1 and the isotype control as described in detail elsewhere (Marx (1992) Lancet 339:707-708). Biopsy tissues were kept at −70° C. as snap-frozen blocks until fresh sections were prepared at the time of experiments. Monoclonal antibodies used for indirect stainings were CD22, CD23, Ki67, CD10, bcl-2 (all DAKO), 4D12 (Smith (1990) Clin Exp Immunol 82:181-187), VS38 (1994) J Clin Pathol 47:418-422). Flow cytometric analysis was performed on a FACScan® (Becton Dickinson) with an Argon ion laser tuned at 488 nm using LYSIS 1I for data acquisition and analysis using triple immunostaining with directly conjugated monoclonal antibodies (CD19 HD 37,Sigma; CD3 UCHT-1,Sigma; CD14; Leu-M3, Becton Dickinson; CD54, 84M10, Immunotech; APO-1, Behrmann (1994) Eur J Immunol 24:3057-3062); CD66L, DREG 56, Dianova; CD40, mAb89 Banchereau (1991) Nature 353:678-679; CD38, AT 13/5, Serotec). Wue-1 was used either directly conjugated with FITC or as biotinylated antibody detected with streptavidin-Phycoerythrin (PE, Sigma). Instrument set-up samples included an un-stained sample, and samples stained with CD19-FITC, CD19-PE and CD19-QR. The instrument set-up was standardized using CD19+ B lymphocytes from normal tonsils as reference by gating on the fluorescence intensity of CD19 lymphocytes followed by adjustment of the light scattering detectors to locate the B cells in a standard po-sition in the correlative display of forward and sideward light scattering. The fluorescence detectors were adjusted using a tight light scattering gate as obtained from the light scattering of the CD19+ lymphocytes, followed by adjustment of the three fluo-rescence detectors of an unstained sample. Each measurement contained 20.000 cells. Dead cell discrimination was performed with 7-amino-actinomycin D (7-AAD, Calbiochem, Germany) in combination with dual color Immunofluorescence as described elsewhere (Schmid (1992) Cytometry 13:204-208).

Analysis of the germinal centers showed that Wue-1 labeling showed strong colocalization with cytoplasmic Ig, surface Ig, Ig isotypes IgM>>IgG>IgA, CD38, VS38, HLA-DR, weak staining could be observed with B cell markers CD19, CD22, CD40; 4D12 (B cells of the marginal zone), CD136, CD95, no staining was with CD23, CD44, CD3 (T cell), CD14 (macrophage), Ki67, CD66L, CD54, CD10, bcl-2, bcl-6, cytokeratin 8, CD68 (macrophage). Differences in the paracortex staining versus the germinal center staining was the absence of surface Ig staining, no CD19, CD22, CD40, CD95, HLA-DR; strong staining was observed with CD136, CD44, CD54, and weak staining with bcl2; all other markers remained unchanged between germinal centers and paracortex. These findings were verified with other lymphatic tissue and bone marrow (data not shown). Furthermore Wue-1 did not cross-react with peripheral blood, liver, heart, muscle, skin, bladder, ovary, testis and the central nervous system, but showed weak staining of secretory epithelia of the gastrointestinal tissues. These results underlined the selective labeling properties of Wue-1.

The ability of Wue-1 to differentiate specific lymphoma subtypes was determined by a number of patient samples analysed as described above. Wue-1 proved to be highly specific for plasma cell malignancies (plasmacytoma/myeloma) and cross-reacting only with immunocytoma. Lymphoma subtypes were classified according to the revised European-American (REAL) classification. Positive staining was observed on 11 of 11 plasmacytoma/myeloma samples; 13 of 13 MALT (mucosa-associated lymphoid tissue)-type lymphoma with plasma cell differentiation but non without plasma cell differentiation (0 of 19). 5 of 6 immunocytoma were Wue-1 positive and 1 of 13 diffuse large cell lymphoma, whereas no staining was observed on follicular center lymphoma (0 of 23), mantle cell lymphoma (0 of 10), Burkitt's lymphoma (0 of 5), B cell lymphocytic leukemia (0 of 5), peripheral T cell lymphoma (0 of 7), angio-immunoblastic lymphoma (0 of 9) and Hodgkin's disease (0 of 13). This highly selective recognition of plasmacytoma/myeloma was not observed with any previously described antibody.

### EXAMPLE 3

### Wue-1 is Suitable for a Highly Selective Antibody-Based Elimination of Plasma Cells

Based on this selective recognition of plasma cells by Wue-1 it is obvious to the person skilled in the art, that such an antibody would be suitable to use in the targeted elimination of malignant plasma cells (plasmacytoma/myeloma). Typical mechanisms used for the antibody-mediated elimination of target cells are targeted delivery of radioactivity (radioimmunoconjugates, DeNardo (1999) Current Opinion in Immunology 11, 563-569), targeted delivery of toxins (immunotoxins, Kreitman (1999) Current Opinion in Immunology 11, 570-578), or antibody-dependent cellular cytotoxicity (ADCC). ADCC can be mediated by Fc-gamma receptor bearing cells (monocytes, macrophages, granulocytes), natural killer cells or T cells. Effector domains connected to the variable domains of an antibody can be the Fc domain, as in a normal monoclonal antibody, or domains that interact with other surface antigens on effector cells.

One promising variant of antibody-based strategies is the bispecific antibody approach by joining two antigen-binding sites of different specificity in one antibody molecule, in order to redirect effector cells to the pre-defined tumor target. Though bispecific antibodies are extremely efficient in recruiting cytotoxic effector cells against tumor cells in vitro and in vivo (Staerz and Bevan (1986). Proc. Natl. Acad. Sci. U.S. A 83, 1453-1457 Lanzavecchia and Scheidegger (1987) Eur. J. Immunol. 17, 105-111.; Kroesen (1995) Cancer Res. 55, 4409-4415; Kroesen (1995) J. Hematother. 4, 409-414;Kroesen (1993) Cancer Immunol. Immunother. 37, 400-407), larger randomized clinical trials proving clinical efficacy are still lacking. The main reason why these promising antibody constructs are dramatically lagging behind intact antibodies in clinical testing, lies in the difficulties to produce sufficient amounts of clinical grade material. Until recently, the production and purification process has been extremely inefficient, with low yields regardless of whether the hybrid-hybridoma approach, chemical linkage or renaturation from bacterial inclusion bodies of recombinant Fab or Fv fragment, was followed. In addition, nearly all of these products are plagued by contamination with ill-defined by-products (Staerz and Bevan, 1986; Lanzavecchia and Scheidegger, 1987; Mallender (1994) Biochemistry 33, 10100-10108; Gruber (1994) J. Immunol. 152, 5368-5374). As previously shown with two different (17-1AxCD3 and CD19xCD3) antibodies, these handicaps can be overcome by the development of a molecular format linking four variable as described in Mack (1995). Proc. Natl. Acad. Sci. U.S. A 92, 7021-7025 and Loeffler (2000) Blood 15, 2098-2103.The resulting recombinant 60 kD molecule produced by mammalian cells is secreted at a high yield in a fully active form that requires no further renaturation.

The variable regions of the Wue-1 monoclonal antibody were cloned using standard PCR-based procedures as described above and the single chain bispecific antibody was assembled from the Wue-1 variable light and heavy chain into to a single-chain antibody and combined with the anti-CD3 single-chain Fv portion of the 17-1AxCD3 antibody via a short peptide linker as described in Mack (1995). The bispecific molecule was designated Wue-1xCD3. Briefly, primers were selected from the 5′ and 3′-end of the coding sequences of the Wue-1 variable regions and were designed to introduce a BsrGl restriction side at the 5′ end of Wue-1-V_{L}, a BspEl restriction side at the 3′ end of Wue-1-V_{H}, and a synthetic (Gly₄Ser₁)₃ linker with an internal BspEl restriction side. Primers were Wue-LC-F: 5′-CTACAGGTGTACACTCCGATATCGTGATGACCCAAACTCC-3′; SEQ ID NO: 17, Wue-1-LC-R: 5′-TCCTCCTCCGGAGCCGCCGCCGCCAGAACCACCACCACCC-CGTTTGATTTCCAGCTTGGTGCC-3′; SEQ ID NO: 18, Wue-1-HC-F: 5′-GGCGGCTCCGGAGGAGGAGATCTC-AGGTGCAGCTGCAGCAGTCTGG-3′; SEQ ID NO: 19, and Wue-1-HC-R: 5′-CCACCACCTCCGGAGGAGACTG-TGAGGGTGGTGCC-3′; SEQ ID NO: 20. Wue-1xCD3 was designed such that the Flag epitope of the 17-1AxCD3 was eliminated. The Wue-1xCD3 bispecific single chain construct into CHO-K1 cells and stable clones were selected using the selection marker dihydrofolate reductase (DHFR). DHFR was linked to the Wue-1xCD3 transcript via an internal ribosomal entry sequence and both expression units were driven by the same EFα-Promotor (Mack (1995)).

The resulting Wue-1xCD3 construct comprised the following nucleotide sequence

(SEQ ID NO: 21)
GAATTCACCA TGGGATGGAG CTGTATCATC CTCTTCTTGG TAGCAACAGC
TACAGGTGTA CACTCCGATA TCGTGATGAC CCAAACTCCA CTCACTTTGT
CGGTTACCAT TGGACAACCA GCCTCCCTCT CTTGCAAGTC AAGTCAGAGC
CTCTTAGATA GTGATGGAAA GACATATTTG AATTGGTTGT TACAGAGGCC
AGGCCAGTCT CCAAAGCGCC TAATCTCTCT GGTGTCTAAA TTGGACTCTG
GAGTCCCTGA CAGATTCACT GGCAGTGGAT CAGGGACAGA TTTCACACTG
AAAATCAGCA GAGTGGAGGC TGAGGATTTG GGAGTCTATT ATTGCTGGCA
AGGTACACAT CTTCCGTGGA CATTCGGTGG AGGCACCAAG CTGGAAATCA
AACGGGGTGG TGGTGGTTCT GGCGGCGGCG GCTCCGGAGG
AGGAGGATCT CAGGTGCAGC TGCAGCAGTC TGGACCTGAG CTAGTGAAGA
CTGGGGCTTC AGTGAAGATA TCTTGTAAGG GTTCTGGTTA CTCATTCAGT
GGTTACTACA TGCACTGGGT CAAGCAGAGC CATGGAAAGA GGCTTGAGTG
GATTGGATAT ATTAGTGGTT ATAATGGTGA TACTAGGTAT AATCAGAAGT
TCAGGGGCAA GGCCACATTT ATTGTAGACA TATCCTCCAG GACAGCCTAC
ATGCAGTTCA ACAGCCTGAC ATCTGAAGAC TCTGCGGTCT ATTACTGTGC
AAGAGGGGGT TACTACGGCT ACGTGGACTA CTGGGGCCAA GGCACCACCC
TCACAGTCTC CTCCGGAGGT GGTGGATCCG ATATCAAACT GCAGCAGTCA
GGGGCTGAAC TGGCAAGACC TGGGGCCTCA GTGAAGATGT CCTGCAAGAC
TTCTGGCTAC ACCTTTACTA GGTACACGAT GCACTGGGTA AAACAGAGGC
CTGGACAGGG TCTGGAATGG ATTGGATACA TTAATCCTAG CCGTGGTTAT
ACTAATTACA ATCAGAAGTT CAAGGACAAG GCCACATTGA CTACAGACAA
ATCCTCCAGC ACAGCCTACA TGCAACTGAG CAGCCTGACA TCTGAGGACT
CTGCAGTCTA TTACTGTGCA AGATATTATG ATGATCATTA CTGCCTTGAC
TACTGGGGCC AAGGCACCAC TCTCACAGTC TCCTCAGTCG AAGGTGGAAG
TGGAGGTTCT GGTGGAAGTG GAGGTTCAGG TGGAGTCGAC GACATTCAGC
TGACCCAGTC TCCAGCAATC ATGTCTGCAT CTCCAGGGGA GAAGGTCACC
ATGACCTGCA GAGCCAGTTC AAGTGTAAGT TACATGAACT GGTACCAGCA
GAAGTCAGGC ACCTCCCCCA AAAGATGGAT TTATGACACA TCCAAAGTGG
CTTCTGGAGT CCCTTATCGC TTCAGTGGCA GTGGGTCTGG GACCTCATAC
TCTCTCACAA TCAGCAGCAT GGAGGCTGAA GATGCTGCCA CTTATTACTG
CCAACAGTGG AGTAGTAACC CGCTCACGTT CGGTGCTGGG ACCAAGCTGG
AGCTGAAACA TCATCACCAT CATCATTAGT CGAC

and the following amino acid sequence

(SEQ ID NO: 22)
DIVMTQTPLT LSVTIGQPAS LSCKSSQSLL DSDGKTYLNW LLQRPGQSPK
RLISLVSKLD SGVPDRFTGS GSGTDFTLKI SRVEAEDLGV YYCWQGTHLP
WTFGGGTKLE IKRGGGGSGG GGSGGGGSQV QLQQSGPELV KTGASVKISC
KGSGYSFSGY YMHWVKQSHG KRLEWIGYIS GYNGDTRYNQ KFRGKATFIV
DISSRTAYMQ FNSLTSEDSA VYYCARGGYY GYVDYWGQGT TLTVSSGGGG
SDIKLQQSGA ELARPGASVK MSCKTSGYTF TRYTMHWVKQ RPGQGLEWIG
YINPSRGYTN YNQKFKDKAT LTTDKSSSTA YMQLSSLTSE DSAVYYCARY
YDDHYCLDYW GQGTTLTVSS VEGGSGGSGG SGGSGGVDDI QLTQSPAIMS
ASPGEKVTMT CRASSSVSYM NWYQQKSGTS PKRWIYDTSK VASGVPYRFS
GSGSGTSYSL TISSMEAEDA ATYYCQQWSS NPLTFGAGTK LELKHHHHHH

Transfected CHO cells were cultivated in serum-free medium and Wue-1xCD3 was harvested from the supernatant. Wue-1xCD3 was purified from the supernatant in a three step purification, starting with cation exchange chromatography, followed by immobilized metal affinity chromatography (IMAC) and gelfiltration. The purity of the isolated protein was >95% as determined by SDS-PAGE (not shown). Under the conditions applied, the molecule appears in its monomeric form about 54 kD. The final yield of purified protein was ca. 2.5 mg/I cell culture supernatant. All chromatography steps were performed on a Äkta FPLC System (Pharmacia). All chemicals were of research grade and purchased from Sigma (Deisenhofen) or Merck (Darmstadt).

A bispecific Wue-1 antibody mediates efficient antibody-dependent mediated cellular cytotoxicity in patient samples

Peripheral blood mononuclear cells (PBMC) were generated by Ficoll (Seromed/Biochrom, Germany) density gradient centrifugation and stimulated with PHA (1 μg/ml) and IL-2 (60U/ml). PBMC were cultured with RPMI, 20% FCS (Seromed/Biochrom, Germany), penicillin/streptomycin and pyruvate (both Life Technologies, Germany). Patient cells were taken from routine diagnostic specimen after informed consent of the patients and approval by the local ethics commitee. Preparation of primary myeloma cells was according to standard procedures. Briefly, mononuclear cells from bone marrow aspirates of multiple myeloma patients were separated by Ficoll density gradient centrifugation and incubated for 15 min with anti-CD138 coated magnetic beads (Miltenyi Biotec, Germany) at 4° C. Separation of CD138+ cells was carried out in a MACS device (Miltenyi Biotec, Germany) according to the manufacturer's instructions. After purification 70-90% of the cells were CD138+(data not shown). Patient cells were taken from routine diagnostic specimen after informed consent of the patients and approval by the local ethics commitee. Antibody-mediated cellular cytotoxicity (ADCC) was determined by flow-cytometry. Enriched multiple myeloma cells were labelled with the PKH-26 dye (Sigma) according to the manufacturers instructions and cultured with PBMCs, antibody and 2 ng/ml IL-6 (PeproTech, Germany). Upon the according incubation time cells were washed with PBS and stained with Propidiumiodine (PI) (2.5 μg/ml). Analysis was performed on a “Facscalibur” flow cytometer using the “Cell-Quest” software (both Becton Dickinson, Germany). The fraction of living myeloma cells were positive for the PKH-26 dye (measured in FL-2) and negative for Pi measured in FL-3). Dead multiple myeloma cells were positive for both, PKH-26 and PI. PBMCs were negative for PKH-26. Specific cytotoxicity was calculated as 100× (1−(viable multiple myeloma cells in test sample/number of viable multiple myeloma cells in 17-1AxCD3 control samples). Assay was stopped after 6 days or if medium colour turned from red to yellowish.

As shown in FIGS. 3 and 4, multiple myeloma samples were incubated with PBMCs pre-stimulated with PHA/IL-2. In 6 experiments it was possible to obtain autologous peripheral blood samples from the bone marrow donors, and 5 experiments were performed using allogeneic PBMCs from healthy donors. No significant difference was found between samples containing autologous or allogeneic PBMCs. Specificity of the kill was controlled by incubation with a bispecific antibody 17-AxCD3 that binds to an epithelial surface antigen not present on plasma cells. Figure three shows a representative experiment displaying PKH-26 staining in the x-axis and propidium iodide in the y-axis. Wue-1xCD3 shows a significant shift of PKH-26 labeled multiple myeloma cells from PI negative to PI positive indicative for cell lysis. FIG. 4 shows the specific lysis of multiple myeloma cells as a function of antibody concentration.

1. An antibody characterized in that the variable region (Fw(L)) of at least one of its light chains (L-chain) has at least one of the following amino acid sequences or a part thereof:
(FW-1; SEQ ID NO: 1)
DIVMTQTPLTLSVTIGQPASLSC
(CDR-1; SEQ ID NO. 2)
KSSQSLLDSDGKTYLN
(FW-2; SEQ ID NO: 3)
WLLQRPGQSPKRLIS
(CDR-2; SEQ ID NO: 4)
LVSKLDS
(FW-3; SEQ ID NO: 5)
GVPDRFTGSGSGTDFTLKISRVEAEDLGVYYC
(CDR-3; SEQ ID NO: 6)
WQGTHLPWT
and/or
(FW-4; SEQ ID NO: 7)
FGGGTKLEIKR
and/or the variable region (fw(h)) of at least one of its heavy chains (h-chain) possesses at least one of the following amino acid sequences or a part thereof:
(FW-1; SEQ ID NO: 8)
QVQLQQSGPELVKTGASVKISCKGSGYSFS,
(CDR-1; SEQ ID NO: 9)
GYYMH
(FW-2; SEQ ID NO: 10)
WVKQSHGKRLEWIG
(CDR-2; SEQ ID NO: 11)
YISGYNGDTRYNQKFRG
(FW-3; SEQ ID NO: 12)
KATFIVDISSRTAYMQFNSLTSEDSAVYYCAR
(CDR-3; SEQ ID NO: 13)
GGYYGYVDY
and/or
(FW-4; SEQ ID NO: 14)
WGQGTTLTVSS.

2. An antibody, characterized in that it is encoded by a gene which contains the following nucleotide sequence or a part thereof for at least one of its light chains (L-chain):
gaagcacgcg tagatatcgt gatgacccaa actccactca ctttgtcggt taccattgga
caaccagcct ccctctcttg caagtcaagt cagagcctct tagatahtga tggaaagaca tatttgaatt
ggttgttaca gaggccaggc cagtctccaa agcgcctaat ctctctggtg tctaaattgg actctggagt
ccctgacaga ttcactggca gtggatcagg gacagatttc acactgaaaa tcagcagagt
ggaggctgag gatttgggag tctattattg ctggcaaggt acacatcttc cgtggacatt cggtggaggc
accaagctgg aaatcaaacg ggctgatgct gcggccgctg gatccatctt c
and/or which contains the following nucleotide sequences or a part thereof for at least one of its heavy chains (H-chain):
cgccatggcc gcgggattcc ggccatggcg caggtgcagc tgcagcagtc tggacctgag
ctagtgaaga ctggggcttc agtgaagata tcttgtaagg gttctggtta ctcattcagt ggttactaca
tgcactgggt caagcagagc catggaaaga ggcttgagtg gattggatat attagtggtt ataatggtga
tactaggtat aatcagaagt tcaggggcaa ggccacattt attgtagaca tatcctccag
gacagcctac atgcagttca acagcctgac atctgaagac tctgcggtct attactgtgc
aagagggggt tactacggct acgtggacta ctggggccaa ggcaccaccc tcacagtctc
ctcagccaaa acgacaccca agcttgtcta tccactggcc cctggtaatc actgtgcggc cgccg

3. The antibody of claims 1 or 2, characterized in that it is an immunoglobulin G.

4. The antibody of any one of claims 1 to 3, characterized in that both light chains and/or both heavy chains have the same amino acid sequence.

5. The antibody of any one of claims 1 to 4 which is produced by the hybridoma cell line DSM ACC 2441.

6. The antibody of any one of claims 1 to 4, characterized in that the antibody is a multifunctional antibody.

7. The antibody of any one of claims 1 to 6, characterized in that it is an antibody conjugate with proteins, such as toxins or cytokines or antibody fragments or enzymes, or with hormones, fluorescent dyes, biotin and/or radioisotopes.

8. The antibody according to anyone of the preceding claims, characterized in that it is a fusion protein with toxins, enzymes, cytokines and/or hormones.

9. A nucleotide sequence coding for an antibody, characterized in that it contains the following nucleotide sequence or a part thereof:
gaagcacgcg tagatatcgt gatgacccaa actccactca ctttgtcggt taccattgga
caaccagcct ccctctcttg caagtcaagt cagagcctct tagatahtga tggaaagaca tatttgaatt
ggttgttaca gaggccaggc cagtctccaa agcgcctaat ctctctggtg tctaaattgg actctggagt
ccctgacaga ttcactggca gtggatcagg gacagatttc acactgaaaa tcagcagagt
ggaggctgag gatttgggag tctattattg ctggcaaggt acacatcttc cgtggacatt cggtggaggc
accaagctgg aaatcaaacg ggctgatgct gcggccgctg gatccatctt c
and/or the following nucleotide sequence or a part thereof
cgccatggcc gcgggattcc ggccatggcg caggtgcagc tgcagcagtc tggacctgag
ctagtgaaga ctggggcttc agtgaagata tcttgtaagg gttctggtta ctcattcagt ggttactaca
tgcactgggt caagcagagc catggaaaga ggcttgagtg gattggatat attagtggtt ataatggtga
tactaggtat aatcagaagt tcaggggcaa ggccacattt attgtagaca tatcctccag
gacagcctac atgcagttca acagcctgac atctgaagac tctgcggtct attactgtgc
aagagggggt tactacggct acgtggacta ctggggccaa ggcaccaccc tcacagtctc
ctcagccaaa acgacaccca agcttgtcta tccactggcc cctggtaatc actgtgcggc cgccg

10. A nucleotide sequence encoding at least a variable region of an antibody as defined in any one of claims 1 to 9.

11. A vector comprising the nucleotide sequence of claim 9 or 10, wherein said vector comprises, optionally, in combination with the nucleotide sequence of claim 10 the variable region of the other immunoglobulin chain of said antibody.

12. A host cell comprising a nucleotide sequence of claim 9 or 10 or comprising the vector of claim 11.

13. A continuous, stable antibody-producing cell line which is capable of producing an antibody as defined in any one of claims 1 to 10.

14. The cell line of claim 13, wherein said cell line is a hybridoma cell line, preferably the hybridoma cell line having the deposit number DSM ACC 2441.

15. A method for preparing an antibody directed against plasma cells, characterized in that an animal is immunized with B-cells of the plasma cell differentiation line up to and including the lymphoplasmacytoid cell stage and the prepared antibody is isolated in a conventional manner from the blood of the animal.

16. A method for preparing an antibody capable of recognizing plasma cells or a functional fragment or derivative thereof comprising
(a) culturing the cell of claim 12, 13 or 14; and
(b) isolating said antibody, functional fragment or an immunoglobulin chain(s) thereof from the culture.

17. An antigen, characterized in that it binds to and/or is recognized by an antibody according to any one of claims 1 to 8.

18. The antigen of claim 17, characterized in that it is presented by human plasma cells on their surface and has a molecular weight of about 94 kD or of about 55 kD.

19. An epitope of the antigen of claim 18.

20. An antibody, characterized in that it binds and/or recognizes an antigen or an epitope according to any one of claims 17 to 19.

21. A nucleotide sequence encoding at least one variable region of the antibody of claim 20.

22. An antibody, fragment or derivative thereof or an immunoglobulin chain encoded by the nucleotide sequence of claim 10, 11 or 21 or obtainable by the method of claim 15 or 16.

23. A method for preparing an antibody directed against plasma cells, characterized in that an animal is immunized with an antigen or an epitope of any one of claims 17 or 19 and the antibody formed is isolated from the blood of the animal in a conventional manner.

24. The method for preparing antibodies of claims 15 or 16, characterized in that the cells of the immunized animal are used to produce a cell line in a conventional manner which produces this antibody as a monoclonal antibody, in that this cell line is cultured and the antibody produced is isolated.

25. The use of an antibody of any one of claims 1 to 9, 20 or 22 for identifying the corresponding antigen.

26. The use of an antibody of any one of claims 1 to 9, 20 or 22 for specifically labeling plasma cells.

27. The use of an antibody of any one of claims 1 to 9, 20 or 22 for the subsequent preparation of additional antibodies which label plasma cells.

28. A single-chain multifunctional polypeptide comprising
(a) a first domain comprising a binding side of an antibody or an immunoglobulin as defined in any one of claims 1 to 9, 20 or 22; and
(b) a second domain comprising a binding side of an immunoglobulin chain or an antibody specifically recognizing the CD3 antigen.

29. The polypeptide of claim 28, wherein said domains are connected by a polypeptide linker.

30. The polypeptide of claim 28 or 29, wherein said first and/or said second domain mimic or correspond to a V_{H} and a V_{L} region from a natural antibody.

31. The polypeptide of any one of claims 28 to 30, wherein said antibody is a monoclonal antibody, a synthetic antibody, a chimeric antibody or a humanized antibody.

32. The polypeptide of any one of claims 28 to 31, wherein said domain is a single-chain fragment of the variable region of the antibody.

33. The polypeptide of any one of claims 28 to 32 that is a bispecific single-chain antibody.

34. The polypeptide of any one of claims 28 to 33 encoded by the nucleic acid molecule as depicted in SEQ ID NO: 21.

35. The polypeptide of any one of claims 28 to 33 comprising the amino acid sequence as depicted in SEQ ID NO: 22.

36. A polynucleotide which upon expression encodes a polypeptide of any one of claims 28 to 35.

37. A vector comprising the polypeptide of claim 36.

38. A cell transfected with the polynucleotide of claim 36 or the vector of claim 37.

39. A composition comprising the antibody of any one of claims 1 to 9, 20 or 22 or a functional fragment or derivative thereof, the multifunctional polypeptide of any one of claims 28 to 35, the nucleotide sequence/polynucleotide of any one of claims 10, 11, 21 or 36, the vector of claim 37, the cell of claim 12 or 38.

40. The composition of claim 39 which is a pharmaceutical composition optionally further comprising a pharmaceutically acceptable carrier.

41. The composition of claim 39 which is a diagnostic composition optionally further comprising suitable means of detection.

42. The use of an antibody of any one of claims 1 to 9, 20 or 22, the multifunctional polypeptide of any one of claims 28 to 35, the nucleotide sequence/polynucleotide of any one of claims 10, 11, 21 or 36, the vector of claim 37, the cell of claim 12 or 38 for treating autoimmune diseases and/or tumors, for instance multiple myelomas, lymphomas and/or plasmocytomas.

43. The use of an antibody of any one of claims 1 to 9, 20 or 22, the multifunctional polypeptide of any one of claims 28 to 35, the nucleotide sequence/polynucleotide of any one of claims 10, 11, 21 or 36, the vector of claim 37, the cell of claim 12 or 38 for preparing a pharmaceutical for the treatment of autoimmune diseases, tumors, for instance multiple myelomas and/or lymphomas.

44. A kit comprising the antibody of any one of claims 1 to 9, 20 or 22, or a functional fragment or derivative thereof, the antigen of claim 17 or 18, the epitope of claim 19, the multifunctional polypeptide of any one of claims 28 to 35, the nucleotide sequence/polynucleotide of any one of claims 10, 11, 21 or 36, the vector of claim 37, the cell of claim 12 or 38.


SEQUENCE LISTING (22 sequences)

SEQ ID NO: 1 - protein, 23 aa, Artificial Sequence
  Description of Artificial Sequence synthetic hybrid, no natural origin
DIVMTQTPLTLSVTIGQPASLSC

SEQ ID NO: 2 - protein, 16 aa, Artificial Sequence
  Description of Artificial Sequence synthetic hybrid, no natural origin
KSSQSLLDSDGKTYLN

SEQ ID NO: 3 - protein, 15 aa, Artificial Sequence
  Description of Artificial Sequence synthetic hybrid, no natural origin
WLLQRPGQSPKRLIS

SEQ ID NO: 4 - protein, 7 aa, Artificial Sequence
  Description of Artificial Sequence synthetic hybrid, no natural origin
LVSKLDS

SEQ ID NO: 5 - protein, 32 aa, Artificial Sequence
  Description of Artificial Sequence synthetic hybrid, no natural origin
GVPDRFTGSGSGTDFTLKISRVEAEDLGVYYC

SEQ ID NO: 6 - protein, 9 aa, Artificial Sequence
  Description of Artificial Sequence synthetic hybrid, no natural origin
WQGTHLPWT

SEQ ID NO: 7 - protein, 11 aa, Artificial Sequence
  Description of Artificial Sequence synthetic hybrid, no natural origin
FGGGTKLEIKR

SEQ ID NO: 8 - protein, 30 aa, Artificial Sequence
  Description of Artificial Sequence synthetic hybrid, no natural origin
QVQLQQSGPELVKTGASVKISCKGSGYSFS

SEQ ID NO: 9 - protein, 5 aa, Artificial Sequence
  Description of Artificial Sequence synthetic hybrid, no natural origin
GYYMH

SEQ ID NO: 10 - protein, 14 aa, Artificial Sequence
  Description of Artificial Sequence synthetic hybrid, no natural origin
WVKQSHGKRLEWIG

SEQ ID NO: 11 - protein, 17 aa, Artificial Sequence
  Description of Artificial Sequence synthetic hybrid, no natural origin
YISGYNGDTRYNQKFRG

SEQ ID NO: 12 - protein, 32 aa, Artificial Sequence
  Description of Artificial Sequence synthetic hybrid, no natural origin
KATFIVDISSRTAYMQFNSLTSEDSAVYYCAR

SEQ ID NO: 13 - protein, 9 aa, Artificial Sequence
  Description of Artificial Sequence synthetic hybrid, no natural origin
GGYYGYVDY

SEQ ID NO: 14 - protein, 11 aa, Artificial Sequence
  Description of Artificial Sequence synthetic hybrid, no natural origin
WGQGTTLTVSS

SEQ ID NO: 15 - DNA, 381 bp, Artificial Sequence
  Description of Artificial Sequence synthetic hybrid, no natural origin
gaagcacgcgtagatatcgtgatgacccaaactccactcactttgtcggttaccattggacaaccagcctccctctcttgcaagtcaagtcagagcctcttagatahtgatggaaagacatatttgaattggttgttacagaggccaggccagtctccaaagcgcctaatctctctggtgtctaaattggactctggagtccctgacagattcactggcagtggatcagggacagatttcacactgaaaatcagcagagtggaggctgaggatttgggagtctattattgctggcaaggtacacatcttccgtggacattcggtggaggcaccaagctggaaatcaaacgggctgatgctgcggccgctggatccatcttc

SEQ ID NO: 16 - DNA, 445 bp, Artificial Sequence
  Description of Artificial Sequence synthetic hybrid, no natural origin
cgccatggccgcgggattccggccatggcgcaggtgcagctgcagcagtctggacctgagctagtgaagactggggcttcagtgaagatatcttgtaagggttctggttactcattcagtggttactacatgcactgggtcaagcagagccatggaaagaggcttgagtggattggatatattagtggttataatggtgatactaggtataatcagaagttcaggggcaaggccacatttattgtagacatatcctccaggacagcctacatgcagttcaacagcctgacatctgaagactctgcggtctattactgtgcaagagggggttactacggctacgtggactactggggccaaggcaccaccctcacagtctcctcagccaaaacgacacccaagcttgtctatccactggcccctggtaatcactgtgcggccgccg

SEQ ID NO: 17 - DNA, 40 bp, Artificial Sequence
  Description of Artificial Sequence synthetic, no natural origin
ctacaggtgtacactccgatatcgtgatgacccaaactcc

SEQ ID NO: 18 - DNA, 63 bp, Artificial Sequence
  Description of Artificial Sequence synthetic, no natural origin
tcctcctccggagccgccgccgccagaaccaccaccaccccgtttgatttccagcttggtgcc

SEQ ID NO: 19 - DNA, 46 bp, Artificial Sequence
  Description of Artificial Sequence synthetic, no natural origin
ggcggctccggaggaggagatctcaggtgcagctgcagcagtctgg

SEQ ID NO: 20 - DNA, 35 bp, Artificial Sequence
  Description of Artificial Sequence synthetic, no natural origin
ccaccacctccggaggagactgtgagggtggtgcc

SEQ ID NO: 21 - DNA, 1574 bp, Artificial Sequence
  Description of Artificial Sequence synthetic, no natural origin
gaattcaccatgggatggagctgtatcatcctcttcttggtagcaacagctacaggtgtacactccgatatcgtgatgacccaaactccactcactttgtcggttaccattggacaaccagcctccctctcttgcaagtcaagtcagagcctcttagatagtgatggaaagacatatttgaattggttgttacagaggccaggccagtctccaaagcgcctaatctctctggtgtctaaattggactctggagtccctgacagattcactggcagtggatcagggacagatttcacactgaaaatcagcagagtggaggctgaggatttgggagtctattattgctggcaaggtacacatcttccgtggacattcggtggaggcaccaagctggaaatcaaacggggtggtggtggttctggcggcggcggctccggaggaggaggatctcaggtgcagctgcagcagtctggacctgagctagtgaagactggggcttcagtgaagatatcttgtaagggttctggttactcattcagtggttactacatgcactgggtcaagcagagccatggaaagaggcttgagtggattggatatattagtggttataatggtgatactaggtataatcagaagttcaggggcaaggccacatttattgtagacatatcctccaggacagcctacatgcagttcaacagcctgacatctgaagactctgcggtctattactgtgcaagagggggttactacggctacgtggactactggggccaaggcaccaccctcacagtctcctccggaggtggtggatccgatatcaaactgcagcagtcaggggctgaactggcaagacctggggcctcagtgaagatgtcctgcaagacttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctcagtcgaaggtggaagtggaggttctggtggaagtggaggttcaggtggagtcgacgacattcagctgacccagtctccagcaatcatgtctgcatctccaggggagaaggtcaccatgacctgcagagccagttcaagtgtaagttacatgaactggtaccagcagaagtcaggcacctcccccaaaagatggatttatgacacatccaaagtggcttctggagtcccttatcgcttcagtggcagtgggtctgggacctcatactctctcacaatcagcagcatggaggctgaagatgctgccacttattactgccaacagtggagtagtaacccgctcacgttcggtgctgggaccaagctggagctgaaacatcatcaccatcatcattagtcgac

SEQ ID NO: 22 - protein, 500 aa, Artificial Sequence
  Description of Artificial Sequence synthetic hybrid, no natural origin
DIVMTQTPLTLSVTIGQPASLSCKSSQSLLDSDGKTYLNWLLQRPGQSPKRLISLVSKLDSGVPDRFTGSGSGTDFTLKISRVEAEDLGVYYCWQGTHLPWTFGGGTKLEIKRGGGGSGGGGSGGGGSQVQLQQSGPELVKTGASVKISCKGSGYSFSGYYMHWVKQSHGKRLEWIGYISGYNGDTRYNQKFRGKATFIVDISSRTAYMQFNSLTSEDSAVYYCARGGYYGYVDYWGQGTTLTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH